(19) United States
(12) Patent Application Publication
(10) Pub. No.: US 2008/0279819 A1
(43) Pub. Date: Nov. 13, 2008

(54) Combinations Therapy for Treatment of Demyelinating Conditions

(75) Inventors: Gregory T. Went, Mill Valley, CA (US); Timothy J. Fultz, Pleasant Hill, CA (US); Laurence R. Meyerson, Las Vegas, NV (US); David Chernoff, San Anselmo, CA (US)

Correspondence Address: Adamas Pharmaceuticals, Inc., 1900 Powell Street, Suite 1050, Emeryville, CA, 94608, US

(73) Assignee: Adamas Pharmaceuticals, Inc.

(21) Appl. No.: 11/884,516

(22) PCT Filed: Feb. 15, 2006
(86) PCT No.: PCT/US06/05540

§ 371 Date: Mar. 18, 2008

Related U.S. Application Data
(60) Provisional application No. 60/653,468, filed on Feb. 15, 2005.

(51) Int. Cl. A61K 39/395; A61K 31/13; A61K 38/21; A61K 31/343; A61K 31/41; A61P 21/00; A61P 25/00; A61P 13/10; A61P 37/00; A61K 31/40; A61K 31/65; A61K 38/07

(52) U.S. Cl. 424/85.6; 514/662; 514/18; 424/133.1; 514/154; 514/470; 514/362; 514/423

(57) ABSTRACT

The present invention provides novel methods and compositions for the treatment and prevention of demyelinating conditions. One demyelinating condition treated by the methods and compositions of the invention is multiple sclerosis. Also treated are symptoms associated with multiple sclerosis.

### FIELD OF THE INVENTION

This invention relates to compositions and methods comprising an uncompetitive NMDA receptor channel antagonist and a multiple sclerosis agent for treatment of demyelinating conditions, such as multiple sclerosis.

### BACKGROUND OF THE INVENTION

Multiple sclerosis (MS) is a progressive central nervous system (CNS) disease that affects over 250,000 Americans. MS is characterized by neuron deterioration in the central nervous system with the associated loss of the insulating myelin sheath from around the axons of the nerve cells (demyelination). This loss of myelin results in loss of electrical insulation and the “short-circuiting” of the electrical pathways mediated by the affected nerves and progressive neurological impairment.

In multiple sclerosis patches of myelin are destroyed by the body's own immune system via a chronic inflammatory autoimmune reaction. This destruction leads to scarring and damage to the underlying nerve fibers, and may manifest itself in a variety of symptoms, depending on the parts of the brain and spinal cord that are affected.

The symptoms associated with MS include pain and tingling in the arms and legs; localized and generalized numbness, muscle spasm and weakness; bowel and bladder dysfunction; difficulty with balance when walking or standing; and fatigue. In most cases, people afflicted with MS lose the ability to stand and/or walk entirely. Optic neuritis may occur episodically throughout the course of the disease. The symptoms are exacerbated by physical fatigue or emotional stress.

Approximately half the people with this disease have relapsing-remitting MS in which there are unpredictable attacks where the clinical symptoms become worse (exacerbation) which are separated by periods of remission where the symptoms stabilize or diminish. The other half have chronic progressive MS without periods of remission.

When flare-ups and exacerbations in MS occur, patients are often treated with high doses of oral or intravenous steroids which may temporarily ameliorate some of the multiple sclerosis symptoms. The gradual nervous system deterioration persists despite this treatment.

A related condition for which there is a long felt need for a non-stimulant pharmacological therapy is the fatigue associated with multiple sclerosis (MS). In one study involving 656 patients with MS, 78% complained of fatigue, 60% experienced it every day, and 22% suffered disruption of their daily activities (Freal et al., Arch. Phys. Med. Rehabil. 65:135, 1984). The National Multiple Sclerosis Society evaluated 839 patients who had only minor neurologic impairment despite having had MS for longer than 10 years, and fatigue was the most commonly reported symptom in this group of mildly affected patients (Jones, New York: National Multiple Sclerosis Society, Health Services Research Report, 1991). In another study 40% of MS patients listed fatigue as the most serious symptom of their disease (Murray, Can. J. Neurol. Sci. 12:251, 1985). Fatigue is reported to be the cause of at least temporary disability in up to 75% of patients with MS.

Amantadine has been used to treat MS related fatigue. Although the mechanism of MS fatigue is poorly understood it has been attributed to nerve conduction abnormalities within the central nervous system and increased energy demands caused by neurologic disability. Several characteristics of MS fatigue are interference with physical functioning and activities of daily living, aggravation by heat, and worsening at the end of the day. Medications that are prescribed for the treatment of MS fatigue include amantadine, pemoline, and other stimulants. Amantadine has been demonstrated to benefit MS fatigue in 79% of patients in a double blind, randomized study, but its mechanism of beneficial action is not known (Krupp et al., Neurology 45:1956, 1995). Although amantadine has been demonstrated in a rigorous fashion to benefit MS fatigue, the benefit is partial for most patients and there are still significant numbers of patients who report no benefit.

More generally, uncompetitive NMDA receptor channel antagonists like memantine (EBIXA™) are known to be neuroprotective, with their action being felt on neurons in an excitotoxic state caused by elevated glutamate, the primary excitatory neurotransmitter. Excessive glutamate can also lead to increased risk of neuronal apoptosis, which is thought to contribute to progress in MS and other neurodegenerative indications. Recently, the United States FDA has approved memantine (NAMENDA™) for use in treating moderate to severe dementia of the Alzheimer's type.

Several general therapeutic approaches have been tried to limit the immune-mediated CNS damage in MS, including antigen-non-specific immunosuppressive drugs and treatments; antigen-specific immunosuppressive drugs and treatments; and cytokine-specific therapies. Some current monotherapies for multiple sclerosis include corticosteroid drugs such as methylprednisolone (SOLUMEDROL™) to alleviate the symptoms of acute episodes, muscle relaxants such as tizanidine hydrochloride (ZANAFLEX™), as well as other biomolecules such as glatiramer acetate (COPAXONE™), mitoxantrone (NOVANTRONE™). In particular, β-interferons (IFN-β) have been tested and approved by the U.S. Food and Drug Administration (FDA) as an MS therapy, e.g., interferon-β1a (AVONEX™, REBIF™) or interferon-β1b (BETASERON™). Other drugs, e.g. τ-interferon (see, e.g., U.S. Pat. No. 6,060,450), vitamin D analogs, e.g., 1.25 (OH)₂D₃ (see, e.g., U.S. Pat. No. 5,716,946), IFN-β-2 (U.S. Patent Publication No. 20020025304), spirogermaniums, (see, e.g., U.S. Pat. No. 4,654,333), prostaglandins, e.g., latanoprost, brimonidine, PGE1, PGE2 or PGE3. (see, e.g., U.S. Patent Publication No. 20020004525), tetracyclines and derivatives thereof, e.g., minocycline, doxycycline (U.S. Patent Publication No. 20020022608), as well as mycophenolic acid (MYFORTIC™) and statins such as atorvastatin (LIPITOR™) are known.

Prior to the discovery of memantine, the available uncompetitive NMDA receptor channel antagonists had been noncompetitive glutamate receptor modulators such that the period of their antagonism was relatively long. Considerable undesirable side effects were seen in which these drugs produced prolonged cognitive impairments including delirium, psychosis and coma. Memantine, a glutamate receptor modulator acts as an un-competitive, use-dependent, pathologically induced glutamate receptor blocker. Memantine is generally well tolerated, with less incidence of delirium, psychosis and cognitive deficits, although adverse effects of administration of memantine are well known (e.g. see Wilcock et al. Int Clin Psychopharmacol 17:297-305 (2002)).

Interferons are known to affect a variety of cellular functions, including DNA replication, and RNA and protein synthesis, in both normal and abnormal cells. Cytotoxic effects of interferon can be manifested in normal, healthy cells. As a result, undesirable side effects may arise during interferon therapy, particularly when high doses are required. Administration of interferon can lead to myelosuppression, thereby resulting in reduced red blood cell count, and reduced white blood cell and platelet levels. Interferons commonly give rise to flu-like symptoms (e.g., fever, fatigue, headaches and chills), gastrointestinal disorders (e.g., anorexia, nausea and diarrhea), dizziness and coughing. Often, the sustained response of patients to interferon treatment is low and the treatment can induce severe side effects, including, but not limited to, retinopathy, thyroiditis, acute pancreatitis, and depression.

Other active agents used in the treatment or demyelinating conditions or associated symptoms also have significant limitations. For example, due to side effects, including asthenia, somnolence, and dizziness, a dose escalation regimen is recommended for tizanidine hydrochloride. Administration of mycophenolate mofetil is associated with increased susceptibility to infection, risk of lymphoma, and side effects including diarrhea, leucopenia, sepsis, and vomiting. Anorexia, nausea, vomiting, and diarrhea are among the side effects arising from doxycycline therapy.

Thus, a need exists to maintain or improve the therapeutic benefits of such therapies while reducing or eliminating the undesirable side effects.

### SUMMARY OF THE INVENTION

The present invention provides a more effective method of treatment for multiple sclerosis (MS) and other demyelinating conditions or symptoms associated therewith, and pharmaceutical compositions which may be used in such methods. The present application addresses both the side effects associated with most MS treatments by a combination of dose reduction and/or reformulation and increased neuroprotection brought about by the addition of an uncompetitive NMDA receptor channel antagonist.

Demyelinating conditions include, for example, multiple sclerosis (MS); progressive multifocal leukoencephalopathy (PML); disseminated necrotizing leukoencephalopathy (DNL); acute disseminated encephalomyelitis; Schilder disease, central pontine myelinolysis (CPM); radiation necrosis; and Binswanger disease (SAE); Guillain-Barre Syndrome; leukodystrophy; acute disseminated encephalomyelitis (ADEM); acute transverse myelitis; acute viral encephalitis; adrenoleukodystrophy (ALD); adrenomyeloneuropathy; AIDS-vacuolar myelopathy; experimental autoimmune encephalomyelitis (EAE); experimental autoimmune neuritis (EAN); HTLV-associated myelopathy; Leber's hereditary optic atrophy; subacute sclerosing panencephalitis; and tropical spastic paraparesis.

In an embodiment, the invention relates to methods for treating multiple sclerosis or other demyelinating condition through the administration of one or more aminoadamantane-derived uncompetitive NMDA receptor channel antagonists, such as memantine, rimantadine, and amantadine in combination with one or more multiple sclerosis agents or treatments, such as interferon-βs (e.g., BETASERON™, REBIF™, or AVONEX™), or other MS agents, such COPAXONE™, ANTEGREN™, NOVANTRONE™, ZENEPAX™, or ZANAFLEX™, SOLUMEDROL™, MYFORTIC™, LIPTOR™, minocycline, or doxycycline. The invention encompasses administration of an uncompetitive NMDA receptor channel antagonist and a multiple sclerosis agent to a subject having multiple sclerosis or other demyelinating condition, such that the multiple sclerosis or other demyelinating condition is treated or at least partially alleviated. The uncompetitive NMDA receptor channel antagonist and multiple sclerosis agent may be administered as part of a pharmaceutical composition, or as part of a combination therapy. In another embodiment, a patient is diagnosed, e.g., to determine if treatment is necessary, whereupon a combination therapy in accordance with the invention is administered to treat the patient.

In an embodiment, the invention relates to methods for treating symptoms associated with multiple sclerosis or other demyelinating condition through the administration of one or more uncompetitive NMDA receptor channel antagonists, such as memantine, rimantadine, and amantadine in combination with multiple sclerosis agents or treatments, such as interferon-βs (e.g., BETASERON™, REBIF™, or AVONEX™), or other MS agents, such COPAXONE™, ANTEGREN™, NOVANTRONE™, ZENEPAX™, or ZANAFLEX™, SOLUMEDROL™, MYFORTIC™, LIPITOR™, minocycline, or doxycycline. In this embodiment, an uncompetitive NMDA receptor channel antagonist and a multiple sclerosis agent are administered to a subject having multiple sclerosis or other demyelinating condition or a symptom associated therewith, such that the multiple sclerosis or other demyelinating condition or symptom is treated or at least partially alleviated.

Symptoms associated with, or arising from, multiple sclerosis, include fatigue, pain and tingling in the arms and legs; localized and generalized numbness, muscle spasm and weakness; bowel and bladder dysfunction; and difficulty with balance when walking or standing. The amount of uncompetitive NMDA receptor channel antagonist and/or a multiple sclerosis agent is typically effective to reduce symptoms and to enable an observation of a reduction in symptoms

The present invention also provides for compositions which include amino-adamantane-derived uncompetitive NMDA receptor channel antagonist agents in combination with multiple sclerosis agents, and are used in the treatment of patients suffering from MS or other demyelinating condition or one or more symptoms associated with MS or another demyelinating condition.

The uncompetitive NMDA receptor channel antagonist agents and multiple sclerosis agent may be administered as part of a pharmaceutical composition, or as part of a combination therapy. In another embodiment, a patient is diagnosed, e.g., to determine if treatment is necessary, whereupon a combination therapy in accordance with the invention is administered to treat the patient. The amount of uncompetitive NMDA receptor channel antagonist agent and a multiple sclerosis agent is typically effective to reduce symptoms and to enable an observation of a reduction in symptoms.

The NMDA receptor antagonist, the second agent (MS agents as used herein), or both agents may be administered in an amount similar to that typically administered to subjects. Optionally, the amount of the NMDA receptor antagonist, the second agent, or both agents may be administered in an amount greater than or less than the amount that is typically administered to subjects. If desired, the amount of the NMDA receptor antagonist in the pharmaceutical composition is less than the amount of NMDA receptor antagonist required in a unit dose to obtain the same therapeutic effect when the NMDA receptor antagonist is administered in the absence of the second agent. Alternatively, the amount of the second agent in the pharmaceutical composition is less than the amount of the second agent required in a unit dose to obtain the same therapeutic effect for treating MS or other demyelinating condition or reducing the symptoms associated therewith when the second agent is administered in the absence of the NMDA receptor antagonist. Optionally, the NMDA receptor antagonist, the second agent, or both are present at a higher dose than that typically administered to a subject for a specific condition due to the combination and/or the use of controlled release methods or materials contemplated herein. For example, the amount of memantine required to positively affect the patient response (inclusive of adverse effects) may be 2.5-80 mg per day rather than the typical 10-20 mg per day administered without the improved formulations described herein. Optionally, lower or reduced amounts of both the NMDA receptor antagonist and the second agent are used in a unit dose relative to the amount of each agent when administered as a monotherapy.

The invention also provides a pharmaceutical composition that includes an NMDA receptor antagonist, a second agent which is an MS agent, and, optionally, a pharmaceutically acceptable carrier. The NMDA receptor antagonist, the second agent, or both agents may be provided in a controlled or extended release form with or without an immediate release component in order to maximize the therapeutic benefit of each, while reducing unwanted side effects associated with each. When these drugs are provided in an oral form without the benefit of controlled or extended release components, they are released and transported into the body fluids over a period of minutes to several hours. Thus, the composition of the invention may contain an NMDA receptor antagonist and a sustained release component, such as a coated sustained release matrix, a sustained release matrix, or a sustained release bead matrix. In one example, memantine (e.g., 5-80 mg) is formulated without an immediate release component using a polymer matrix (e.g., Eudragit), Hydroxypropyl methyl cellulose (HPMC) and a polymer coating (e.g., Eudragit). Such formulations are compressed into solid tablets or granules. Optionally, a coating such as Opadry® or Surelease® is used.

Optionally, the compositions described herein is formulated such that the NMDA receptor antagonist or the second agent has an in vitro dissolution profile less than 40% in one hour, less than 70% in four hours, between 1% and 80% in 6 hours, 30% and 90% in 10 hours, and 60% and 100% in 12 hours and greater than 84% in 16 hours using, for example, a USP type 2 (paddle) dissolution system at 50 rpm at a temperature of 37±0.5° C. with 0.1N HCl as a dissolution medium. Alternatively the NMDA receptor antagonist or the second agent has an in vitro dissolution profile in a solution with a neutral pH (e.g., water) that is substantially the same as its dissolution profile in an acidic dissolution medium.

Optionally, the composition described herein is formulated such the N-methyl-D-aspartate (NMDA) receptor antagonist or the second agent has an in vitro dissolution profile ranging between 0.1%-20% in one hour, 5%-30% in two hours, 40%-80% in six hours, 50%-90% in 10 hours, and 90%-95% in 12 hours using, for example, a USP type 2 (paddle) dissolution system at 50 rpm, at a temperature of 37±0.5° C. with 0.1N HCl as a dissolution medium. Alternatively, the N-methyl-D-aspartate (NMDA) receptor antagonist or the second agent has an in vitro dissolution profile in a solution with a neutral pH (e.g., water) that is substantially the same as its dissolution profile in an acidic dissolution medium. Thus, the NMDA receptor antagonist or the second agent may be released at the following rate: between 0.1-20% in one hour, 5-30% in two hours, 40-80% in six hours, 70-90% in 10 hours, and 90%-95% in 12 hours as obtained using a USP type II (paddle) dissolution system at 50 rpm, at a temperature of 37±0.5° C.

Desirably, the compositions described herein have an in vitro profile that is substantially identical to the dissolution profile shown for the controlled release formulations shown in the figures and, upon administration to a subject at a substantially constant daily dose, achieves a serum concentration profile that is substantially identical to that shown in the figures.

As used herein, “C” refers to the concentration of an active pharmaceutical ingredient in a biological sample, such as a patient sample (e.g. blood, serum, and cerebrospinal fluid). The concentration of the drug in the biological may be determined by any standard assay method known in the art. The term “Cmax” refers to the maximum concentration reached by a given dose of drug in a biological sample. The term “Cmean” refers to the average concentration of the drug in the sample over time. Cmax and Cmean may be further defined to refer to specific time periods relative to administration of the drug. The time required to reach the maximal concentration (“Cmax”) in a particular patient sample type is referred to as the “Tmax.” The agents of the combination are administered in formulations that reduce the variability of the ratio of the concentrations of the active agents over a period of time, thereby maximizing the therapeutic benefit while minimizing the side effects.

If desired, the dosage form is provided in a non-dose escalating, twice per day or once per day form. In such cases, the concentration ramp (or Tmax effect) may be reduced so that the change in concentration as a function of time (“dC/dT”) is altered to reduce or eliminate the need to dose escalate the drug. A reduction in dC/dT may be accomplished, for example, by increasing the Tmax in a relatively proportional manner. Accordingly, a two-fold increase in the Tmax value may reduce dC/dT by approximately a factor of two. Thus, the NMDA receptor antagonist may be provided so that it is released at a dC/dT that is significantly reduced over an immediate release (so called IR) dosage form, with an associated delay in the Tmax. Thus, the NMDA receptor antagonist may be provided so that it is released at a rate that is significantly reduced over an immediate release (so called IR) dosage form, with an associated delay in the Tmax. The pharmaceutical composition may be formulated to provide a shift in Tmax by 24 hours, 16 hours, 8 hours, 4 hours, 2 hours, or at least 1 hour. The associated reduction in dC/dT may be by a factor of approximately 0.05, 0.10, 0.25, 0.5, or at least 0.8. In certain embodiments, this is accomplished by releasing less than 30%, 50%, 75%, 90%, or 95% of the NMDA receptor antagonist into the circulatory or neural system within one hour of such administration.

Optionally, the sustained release formulations exhibit plasma concentration curves having initial (e.g., from 2 hours after administration to 4 hours after administration) slopes less than 75%, 50%, 40%, 30%, 20% or 10% of those for an IR formulation of the same dosage of the same NMDA receptor antagonist. The precise slope for a given individual will vary according to the NMDA receptor antagonist being used, the quantity delivered, or other factors, including, for some active pharmaceutical agents, whether the patient has eaten or not. For other doses, e.g., those mentioned above, the slopes vary directly in relationship to dose. The determination of initial slopes of plasma concentration is described, for example, in U.S. Pat. No. 6,913,768, hereby incorporated by reference.

Using the sustained release formulations described herein, the NMDA receptor antagonist or the second agent reaches a therapeutically effective steady state plasma concentration in a subject within the course of the first five, seven, nine, ten, twelve, fifteen, or twenty days of administration. For example, the formulations described herein, when administered at a substantially constant daily dose (e.g., at a dose ranging between 15 mg and 35 mg and preferably between 20 and 25 mg) may reach a steady state plasma concentration in approximately 70%, 60%, 50%, 40%, 30%, or less of the time required to reach such plasma concentration when using a dose escalating regimen.

The ratio of the concentrations of two agents in a combination is referred to as the “Cratio,” which may fluctuate as the combination of drugs is released, transported into the circulatory system or CNS, metabolized, and eliminated. An objective of the present invention is to stabilize the Cratio for the combinations described herein. In some embodiments, the variation in the Cratio (termed “Cratio,var”) is as low as possible.

The present invention therefore features formulations of combinations directed to dose optimization or release modification to reduce adverse effects associated with separate administration of each agent. The combination of the NMDA receptor antagonist and the second agent may result in an additive or synergistic response, as described below.

If desired, the NMDA receptor antagonist is released into a subject sample at a slower rate than observed for an immediate release (IR) formulation of the same quantity of the antagonist. The release rate is measured as the dC/dT over a defined period within the period of 0 to Tmax. For the IR formulation the dC/dT rate is less than about 80% of the rate for the IR formulation. In some embodiments, the dC/dT rate is less than about 60%, 50%, 40%, 30%, 20%, or 10% of the rate for the IR formulation. Similarly, the second agent may also be released into a patient sample at a slower rate than observed for an IR formulation of the same quantity wherein the release rate is measured as the dC/dT over a defined period within the period of 0 to Tmax for the IR formulation and the dC/dT rate is less than about 80%, 60%, 50%, 40%, 30%, 20%, or 10%, of the rate for the IR formulation.

In all foregoing aspects of the invention, at least 50%, 80, 90%, 95%, or essentially all of the NMDA receptor antagonist in the pharmaceutical composition may be provided in a controlled release dosage form. In some embodiments, at least 99% of the NMDA receptor antagonist remains in the extended dosage form one hour following introduction of the pharmaceutical composition into a subject. The NMDA receptor antagonist may have a Cₘₐₓ/Cₘₑₐₙ of approximately 2, 1.6, 1.5, 1.4, 1.3, 1.2 or less, approximately 2 hours to at least 8, 12, 16, 24 hours after the NMDA receptor antagonist is introduced into a subject. The second agent may also be provided in a controlled release dosage form. Thus, at least 50%, 60%, 70%, 80%, 90%, 95%, or essentially all of the second agent may be provided as a controlled release formulation. If provided as such, the second agent may have a Cₘₐₓ/Cₘₑₐₙ of approximately 2, 1.6, 1.5, 1.4, 1.3, 1.2 or less, approximately 2 hours to at least 6, 8, 12, 16, or 24 hours after the second agent is introduced into a subject.

The active pharmaceutical agents may be administered to the patient in a manner that reduces the variability of the ratio of the concentrations of the active agents over a period of time, thereby maximizing the therapeutic benefit while minimizing the side effects. The present invention differs from prior studies by providing novel combinations as well as formulations of combinations directed to dose optimization or release modification to reduce adverse effects associated with each agent.

Optionally, the Cratio,var of the NMDA receptor antagonist and the second agent is less than 100%, e.g., less than 70%, 50%, 30%, 20%, or 10% after the agents have reached steady-state conditions. Optionally, the Cratio,var of the NMDA receptor antagonist and the second agent is less than 100%, e.g. less than 70%, 50%, 30%, 20%, or 10% during the first 24 hours post-administration of the agents. In some embodiments, the Cratio,var is less than about 90% (e.g., less than about 75% or 50%) of that for IR administration of the same active pharmaceutical ingredients over the first 4, 6, 8, or 12 hours after administration.

The amino-adamantane-derived uncompetitive NMDA receptor channel antagonist agents which may be used in the invention include memantine (1-amino-3,5-dimethyladamantane), rimantadine (1-(1-aminoethyl)adamantane), or amantadine (1-amino-adamantane), pharmaceutically acceptable salts, and combinations thereof. Other amino-adamantane-derived uncompetitive NMDA receptor channel antagonist agents are those described in U.S. Pat. No. 5,061,703. Generally, for memantine the dosage is from about 5 to about 20, 40, 60, 80 mg/day, for amantadine the dosage is from about 50 to about 200, 400, 600, 800 mg/day, and for rimantadine the dosage is from about 50 to about 200, 400, 600 mg/day. Memantine is particularly preferred as the amino-adamantane-derived uncompetitive NMDA receptor channel antagonist.

The MS agent which may be used in the pharmaceutical compositions, methods and combination therapies of the invention include β-interferons, glatiramer acetate, natalizumab, mitoxanthrone and daclizumab.

In certain embodiments, the multiple sclerosis agent is a β-interferon, e.g., interferon-β1a, interferon-β1b, or interferon-β2; or glatiramer acetate. When interferon-β1a (i.e., AVONEX™) is used, it is administered at a dosage of about 7.5 to about 30 μg preferably intramuscularly, once a week. When interferon-β1a (i.e., REBIF™) is used, it is administered at a dosage of about 11 μg to about 44 μg, preferably subcutaneously and preferably three times a week. When interferon-β1b (e.g., BETASERON™) is used, it is administered at a dosage of about 50 μg to about 250 μg, preferably subcutaneously and preferably every other day. When glatiramer acetate (e.g., COPAXONE™) is used, it is administered at a dosage of about 5 mg to about 20 mg, e.g., 10, 15 or 20 mg, and is preferably administered subcutaneously, preferably daily. When natalizumab (ANTEGREN™) is used, it is administered at a dosage of about 1.5 mg/kg to 6 mg/kg by intravenous infusion, preferably once every four weeks. When mitoxanthrone (NOVANTRONE™) is used for reducing neurologic disability and/or the frequency of clinical relapses in patients with secondary (chronic) progressive, progressive relapsing, or worsening relapsing remitting multiple sclerosis, the recommended dosage of NOVANTRONE is about 3 to about 12 mg/m² given as a short (approximately 5 to 15 minutes) intravenous infusion every 3 months. When daclizumab (ZENAPAX™) is used, it is administered at a dosage of about 0.25 to about 1 mg/kg (intravenous) every 14 days, for a total of 5 doses.

In one embodiment, a combination therapy for MS or other demyelinating condition includes memantine and β-interferon, for treating a patient in need of such treatment. In another embodiment, a combination therapy for MS or other demyelinating condition includes rimantadine and β-interferon, for treating a patient in need of such treatment. In another embodiment, a combination therapy for MS or other demyelinating condition includes amantadine and β-interferon, for treating a patient in need of such treatment.

In another embodiment, a combination therapy for MS or other demyelinating condition includes memantine and glatiramer, for treating a patient in need of such treatment. In one embodiment, a combination therapy for MS or other demyelinating condition includes rimantadine and glatiramer, for treating a patient in need of such treatment. In an embodiment, a combination therapy for MS or other demyelinating condition includes amantadine and glatiramer, for treating a patient in need of such treatment.

In yet another embodiment, a combination therapy for MS or other demyelinating condition includes memantine and natalizumab, for treating a patient in need of such treatment. In one embodiment, a combination therapy for MS or other demyelinating condition includes rimantadine and natalizumab, for treating a patient in need of such treatment. In yet another embodiment, a combination therapy for MS or other demyelinating condition includes amantadine and natalizumab, for treating a patient in need of such treatment.

In yet another embodiment, a combination therapy for MS or other demyelinating condition includes memantine and daclizumab, for treating a patient in need of such treatment. In one embodiment, a combination therapy for MS or other demyelinating condition includes rimantadine and daclizumab, for treating a patient in need of such treatment. In yet another embodiment, a combination therapy for MS or other demyelinating condition includes amantadine and daclizumab, for treating a patient in need of such treatment. In yet another embodiment, a combination therapy for MS or other demyelinating condition includes memantine and mitoxanthrone, for treating a patient in need of such treatment. In one embodiment, a combination therapy for MS or other demyelinating condition includes rimantadine and mitoxanthrone, for treating a patient in need of such treatment. In yet another embodiment, a combination therapy for MS or other demyelinating condition includes amantadine and mitoxanthrone, for treating a patient in need of such treatment.

In some embodiments, the NMDA receptor antagonist, the second agent, or both agents are formulated for oral, intravenous, topical, intranasal, subcutaneous, subtopical transepithelial, subdermal, intramuscular, or inhalation delivery. Thus, the agents described herein may be formulated as a suspension, capsule, tablet, suppository, lotion, patch, or device (e.g., a subdermally implantable delivery device or an inhalation pump). If desired, the NMDA antagonist and the second agent may be admixed in a single composition. Alternatively, the two agents are delivered in separate formulations sequentially, or within one hour, two hours, three hours, six hours, 12 hours, or 24 hours of each other. If administered separately, the two agents may be administered by the same or different routes of administration three times a day, twice a day, once a day, or even once every two days.

Preferably, the NMDA receptor antagonist and the second agent are provided in a unit dosage form.

The invention further relates to kits for treating patients having multiple sclerosis or other demyelinating condition, comprising a therapeutically effective dose of an agent for treating or at least partially alleviating the symptoms of MS or other demyelinating condition (e.g., β-interferons, glatiramer acetate, natalizumab, or daclizumab), and an uncompetitive NMDA receptor channel antagonist, either in the same or separate packaging, and instructions for its use.

Pharmaceutical compositions comprising an uncompetitive NMDA receptor channel antagonist and a multiple sclerosis agent, in effective amount(s) and pharmaceutically acceptable carrier, to treat MS or other demyelinating condition, are also included in the invention.

The above description sets forth rather broadly the more important features of the present invention in order that the detailed description thereof that follows may be understood, and in order that the present contributions to the art may be better appreciated. Other objects and features of the present invention will become apparent from the following detailed description. It is to be understood, however, that the drawings are designed solely for the purposes of illustration and not as a definition of the limits of the invention, for which reference should be made to the appended claims.

### BRIEF DESCRIPTION OF THE FIGURES

FIG. 1A is a graph showing the dissolution profiles of an immediate release formulation of memantine (Namenda) and sustained release formulations of memantine (NPI-6601, NPI-6701, and NPI-6801). The sustained release formulations contain 22.5 mg of memantine. These dissolution profiles were obtained from a USP II Paddle system using water as the medium.

FIG. 1B is a graph showing predicted plasma blood levels for 24 hours of dosing with an immediate release formulation of memantine (Namenda) and sustained release formulations of memantine (NPI-6601, NPI-6701, and NPI-6801), obtained using the Gastro-Plus software package v.4.0.2. The sustained release formulations contain 22.5 mg of memantine.

FIG. 2A is a graph showing the dissolution profiles for Tizanidine sustained release (SR) Systems (120 mg) component of combination, obtained using the Gastro-Plus software package v.4.0.2

FIG. 2B is a graph showing the predicted plasma blood levels for 24 hours of dosing with Tizanidine IR and SR Systems component of combination, obtained using the Gastro-Plus software package v.4.0.2.

FIG. 2C is a graph showing the predicted plasma blood levels over 24 hours of dosing with Memantine and Tizanidine SR combination, obtained using the Gastro-Plus software package v.4.0.2.

FIG. 2D is a graph showing the predicted plasma blood levels over multiple dosing using present NPI Memantine SR and Tizanidine SR combinations, obtained using the Gastro-Plus software package v.4.0.2.

FIG. 3 is a graph showing the predicted plasma blood levels over 24 hours of dosing with memantine and atorvastatin combination.

FIG. 4A is graph showing simulated dissolution profiles for Doxycycline SR systems (120 mg) component of the combination.

FIG. 4B is a graph showing predicted plasma blood levels over 24 hours of dosing with Doxycycline SR Systems component of the combination, obtained using the Gastro-Plus software package v.4.0.2.

FIG. 4C is a graph showing predicted plasma blood levels over 24 hours with Memantine SR and Doxycycline SR combination.

FIG. 4D is a graph showing the predicted plasma blood levels over multiple dosing using NPI Memantine SR systems and Doxycycline SR combinations.

The features and other details of the invention will now be more particularly described and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified. The scientific publications, patents or patent applications cited in the various sections of this document are herein incorporated-by-reference for all purposes.

### DETAILED DESCRIPTION OF THE INVENTION

The present invention provides methods and compositions for treating or preventing demyelinating conditions, such as multiple sclerosis using an NMDA receptor antagonist (e.g., memantine, amantadine, or rimantadine) and one or more multiple sclerosis agents or treatments, such as interferon-βs (e.g., BETASERON™, REBIF™, or AVONEX™), or other MS agents, such COPAXONE™, ANTEGREN™, NOVANTRONE™, ZENEPAX™, or ZANAFLEX™, SOLUMEDROL™, MYFORTIC™, LIPTOR™, minocycline, or doxycycline. Desirably, either of these two agents, or even both agents, is formulated for extended release, thereby providing a concentration and optimal concentration ratio over a desired time period that is high enough to be therapeutically effective but low enough to reduce or avoid adverse events associated with excessive levels of either agent in the subject.

As used herein, the term “Agent” includes a protein, polypeptide, peptide, nucleic acid (including DNA or RNA), antibody, molecule, compound, antibiotic, or drug, and any combinations thereof.

“Treating”, includes any effect, e.g., lessening, reducing, modulating, or eliminating, that results in the improvement of the condition, disease, disorder, etc.

Preferably, the term “subject” refers to a mammal. More preferably, the term subject refers to a primate. More preferably, the term subject refers to a human.

“Multiple Sclerosis Symptoms,” includes the commonly observed symptoms of multiple sclerosis, such as those described in Treatment of Multiple Sclerosis: Trial Design, Results, and Future Perspectives, ed. Rudick and D. Goodkin, Springer-Verlag, New York, 1992, particularly those symptoms described on pages 48-52.

“Pharmaceutically or Pharmacologically Acceptable” include molecular entities and compositions that do not produce an adverse, allergic or other untoward reaction when administered to an animal, or a human, as appropriate.

“Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

“Pharmaceutically Acceptable Salts” include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

“Demyelinating Conditions” include, without limitation, such disorders as multiple sclerosis (MS); progressive multifocal leukoencephalopathy (PML); disseminated necrotizing leukoencephalopathy (DNL); acute disseminated encephalomyelitis; Schilder disease, central pontine myelinolysis (CPM); radiation necrosis; and Binswanger disease (SAE); Guillain-Barre Syndrome; leukodystrophy; acute disseminated encephalomyelitis (ADEM); acute transverse myelitis; acute viral encephalitis; adrenoleukodystrophy (ALD); adrenomyeloneuropathy; AIDS-vacuolar myelopathy; experimental autoimmune encephalomyelitis (EAE); experimental autoimmune neuritis (EAN); HTLV-associated myelopathy; Leber's hereditary optic atrophy; subacute sclerosing panencephalitis; and tropical spastic paraparesis. Such conditions are characterized by loss of myelin. Disruption in the ability of the nerves to conduct electrical impulses to and from the brain often result from such loss of myelin.

“Uncompetitive NMDA receptor channel antagonists” include amino-adamantanes and derivatives thereof. Amino adamantanes and derivatives include adamantane derived or amantadine-derived molecules capable of acting as antagonists of the N-methyl-D-aspartate (NMDA) type receptors, and pharmaceutically acceptable salts and esters thereof. Members of the uncompetitive NMDA receptor channel antagonist family include those described in U.S. Pat. No. 5,061,703. Preferably, the uncompetitive NMDA receptor channel antagonists of the invention are amantadine, memantine, and rimantadine.

The NMDA receptor antagonist may be provided so that it is released at a dC/dT that is significantly reduced over an instant release (so called IR) dosage form, with an associated delay in the Tmax. The pharmaceutical composition may be formulated to provide a shift in Tmax by 24 hours, 16 hours, 8 hours, 4 hours, 2 hours, or at least 1 hour. The associated reduction in dC/dT may be by a factor of approximately 0.05, 0.10, 0.25, 0.5 or at least 0.8. In addition, the NMDA receptor antagonist may be provided such that it is released at rate resulting in a Cₘₐₓ/Cₘₑₐₙ of approximately 2 or less for approximately 2 hours to at least 8 hours after the NMDA receptor antagonist is introduced into a subject. The pharmaceutical composition may be formulated to provide memantine in an amount ranging between 1 and 80 mg/day, 5 and 40 mg/day, 5 and 20 mg/day; or 10 and 20 mg/day; amantadine in an amount ranging between 25 and 500 mg/day, 25 and 300 mg/day, or 100 and 300 mg/day; or dextromethorphan in an amount ranging between 1 and 5000 mg/day, 1 and 1000 mg/day, 100 and 800 mg/day, or 200 and 500 mg/day. Pediatric doses will typically be lower than those determined for adults. Representative dosing can be found in the PDR by anyone skilled in the art.

Table 1 shows exemplary the pharmacokinetic properties (e.g., Tmax and T1/2) of memantine, amantadine, and rimantadine

TABLE 1
Pharmacokinetics and Tox in humans for selected
NMDAr antagonists

| | Human | | | |
|---|---|---|---|---|
| | PK (t½) | Tmax in | Normal | Dose Dependent |
| Compound | in hrs | hrs | Dose | Tox |
| Memantine | 60 | 3 | 10-20 mg/day, | Dose escalation |
| | | | starting at 5 mg | required, |
| | | | | hallucination |
| Amantadine | 15 | 3 | 100-300 mg/day | Hallucination |
| Rimantadine | 25 | 6 | 100-200 mg/day | Insomnia |

“Multiple Sclerosis Agents” include molecules useful for the treatment of multiple sclerosis or other demyelinating diseases or symptoms associated therewith. Examples include β-interferons, glatiramer acetate, natalizumab, mitoxantrone, daclizumab, methylprednisolone, tizanidine hydrochloride, minocycline, doxyxycline, mycophenolic acid, and statins such as atorvastatin.

The multiple sclerosis agent which is used in compositions of the invention is preferably, a β-interferon, e.g., interferon-β1a, interferon-β1b, or interferon-β2 (AVONEX™ BETASERON™ or REBIF™); glatiramer acetate (e.g., COPAXONE™); natalizumab (ANTEGREN™); mitoxanthrone (NOVANTRONE™); daclizumab (ZENAPAX™); tizanidine hydrochloride (ZANAFLEX™), mycophenolic acid (MYFORTIC™), atorvastatin (LIPITOR™), and doxycycline.

The terms “beta interferon”, “beta-interferon”, “beta IFN”, “beta-IFN”, “β interferon”, “β-interferon”, “β IFN”, “β-IFN”, “interferon beta”, “interferon-beta”, “interferon β”, “interferon-β”, “IFN beta”, “IFN-beta”, “IFN β”, “IFN-β”, and “human fibroblast interferon” are used interchangeably herein to describe members of the group of interferon beta's which have distinct amino acid sequences as have been identified by isolating and sequencing DNA encoding the peptides.

Additionally, the terms “beta interferon 1a”, “beta interferon-1a” “beta-interferon 1a”, “beta-interferon-1a”, “beta IFN 1a”, “beta IFN-1a”, “beta-IFN 1a”, “beta-IFN-1a”, “βinterferon 1a”, “β interferon-1a”, “β-interferon 1a”, “β-interferon-1a”, “β IFN 1a”, “β IFN-1a”, “β-IFN 1a”, “β-IFN-1a”, “interferon beta 1a”, “interferon beta-1a”, “interferon-beta 1a”, “interferon-beta-1a”, “interferon β 1a”, “interferon β-1a”, “interferon-β 1a”, “interferon-β-1a”, “IFN beta 1a”, “IFN beta-1a”, “IFN-beta 1a”, “IFN-beta-1a”, “IFN β 1a”, “IFN β-1a”, “IFN-β 1a”, “IFN-β-1a” are used interchangeably herein to describe recombinantly- or synthetically-produced interferon beta that has the naturally-occurring (wild type) amino acid sequences.

The multiple sclerosis agent which is used combination therapies of the invention is preferably, an β-interferon, e.g., interferon-β1a, interferon-β1b, interferon-β2, glatiramer acetate, natalizumab, mitoxanthrone, or daclizumab. When interferon-β1a (i.e., AVONEX™) is used, it is administered at a dosage of about 7.5 μg to 30 μg preferably intramuscularly, once a week. When interferon-β1a (i.e., REBIF™) is used, it is administered at a dosage of about 11 μg to about 44 μg, preferably 22 μg, and most preferably 44 μg, preferably subcutaneously and preferably three times a week. When interferon-β1b (e.g., BETASERON™) is used, it is administered at a dosage of about 50 μg to about 250 μg, preferably subcutaneously and preferably every other day. When glatiramer acetate (e.g., COPAXONE™) is used, it is administered at a dosage of about 5 to about 20 mg preferably administered subcutaneously, preferably daily. When natalizumab (ANTEGREN™) is used, it is administered at a dosage of about 1.5 mg/kg to 6 mg/kg by intravenous infusion, preferably once every four weeks. When daclizumab (ZENAPAX™) is used, it is administered in five doses at about 0.25 to about 1 mg/kg (intravenous) with a 14 day interval between doses. Peak serum concentrations are between 21-32 μg/ml. When mitoxanthrone (NOVANTRONE™) is used for reducing neurologic disability and/or the frequency of clinical relapses in patients with secondary (chronic) progressive, progressive relapsing, or worsening relapsing remitting multiple sclerosis, the recommended dosage of NOVANTRONE™ is about 3 to about 12 mg/m² given as a short (approximately 5 to 15 minutes) intravenous infusion every 3 months.

For example, if IFN-β2 is used in the combination therapies of the invention, it can be administered in effective amounts for treatment, e.g., 1.6 MIU (million International Units according to the international reference standard) and 8 MIU administered subcutaneously on alternate days. Effective amounts can be determined routinely, e.g., by performing a dose-response experiment in which varying doses are administered to target cells to determine an effective amount in achieving the desired purpose. Amounts can be selected based on various factors, including the milieu to which the IFN-β2 is administered (e.g., a patient with multiple sclerosis, animal model, tissue culture cells, etc.), the site of the cells to be treated, the age, health, gender, and weight of a patient or animal to be treated, etc.

Drug Class 1 & 2 Together

TABLE 1
Multiple Sclerosis Dosage Table

| | Multiple Sclerosis Agents | | | | |
|---|---|---|---|---|---|
| NMDA drug | Interferon β-1b/ | Interferon β-1a/ | Interferon β-1a/ | Glatiramer/ | Mitoxantrone/ |
| (mg) | BETASERON ™ | REBIF ™ | AVONEX ™ | COPAXONE ™ | NOVANTRONE ™ |
| Memantine/ | 50-250 μg | 11-44 μg | 7.5-30 μg | 5-20 mg sc/day | 3-12 mg/m²/3 |
| 5-20/day | sc/every other | sc/3/week | IM/week | | months |
| | day | | | | not to exceed |
| | | | | | 140/m sq |
| Amantadine/ | 50-250 μg | 11-44 μg | 7.5-30 μg | 5-20 mg | 3-12 mg/m²/3 |
| 50-200/day | sc/every other | sc/3/week | IM/week | sc/day | months |
| | day | | | | not to exceed |
| | | | | | 140/m sq |
| Rimantadine/ | 50-250 μg | 11-44 μg | 7.5-30 μg | 5-20 mg | 3-12 mg/m²/3 |
| 50-200/day | sc/every other | sc/3/week | IM/week | sc/day | months |
| | day | | | | not to exceed |
| | | | | | 140/m sq |

sc = Subcutaneous injection
IM = Intramuscular injection

TABLE 2
Multiple Sclerosis Dosage Table

| | Multiple Sclerosis Agents | | | |
|---|---|---|---|---|
| | Tizanidine | Atorvastatin/ | Natalizumab/ | Daclizumab/ |
| NMDA drug (mg) | HCL | LIPITOR ™ | ANTEGREN ™ | ZENAPAX ™ |
| Memantine/ | 10-36 mg/day | 10-80 mg/day | 1.5-6 mg/kg sc | 0.25-1 mg/kg |
| 5-20/day | | | or IM, once | sc or IM every |
| | | | every 4 weeks | 14 days, for a |
| | | | | total of 5 doses |
| Amantadine/ | 10-36 mg/day | 10-80 mg/day | 1.5-6 mg/kg sc | 0.25-1 mg/kg |
| 50-200/day | | | or IM, once | sc or IM every |
| | | | every 4 weeks | 14 days, for a |
| | | | | total of 5 doses |
| Rimantadine/ | 10-36 mg/day | 10-80 mg/day | 1.5-6 mg/kg sc | 0.25-1 mg/kg |
| 50-200/day | | | or IM, once | sc or IM every |
| | | | every 4 weeks | 14 days, for a |
| | | | | total of 5 doses |

sc = Subcutaneous injection
IM = Intramuscular injection

“Combination Therapy” (or “co-therapy”) includes the administration of an uncompetitive NMDA receptor channel antagonist and a multiple sclerosis agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Compositions of the invention useful for treating demyelinating conditions, such as MS, include a therapeutic amount of an uncompetitive NMDA receptor channel antagonist and a therapeutic amount of a multiple sclerosis agent. A composition for a demyelinating condition may include memantine and β-interferon. In another embodiment, a composition for a demyelinating condition includes rimantadine and β-interferon. In an embodiment, a composition for a demyelinating condition includes amantadine and β-interferon.

In another embodiment, a composition for a demyelinating condition includes memantine and glatiramer. In one embodiment, a composition for a demyelinating condition includes rimantadine and glatiramer. In an embodiment, a composition for a demyelinating condition includes amantadine and glatiramer.

In yet another embodiment, a composition for a demyelinating condition includes memantine and natalizumab. In another embodiment, a composition for a demyelinating condition includes rimantadine and natalizumab. In yet another embodiment, a composition for a demyelinating condition includes amantadine and natalizumab.

In yet another embodiment, a composition for a demyelinating condition includes memantine and daclizumab. In another embodiment, a composition for a demyelinating condition includes rimantadine and daclizumab. In yet another embodiment, a composition for a demyelinating condition includes amantadine and daclizumab. In a preferred embodiment, the demyelinating condition is MS.

In yet another embodiment, a composition for a demyelinating condition includes memantine and mitoxanthrone. In another embodiment, a composition for a demyelinating condition includes rimantadine and mitoxanthrone. In yet another embodiment, a composition for a demyelinating condition includes amantadine and mitoxanthrone.

In a preferred embodiment, the composition of the invention is memantine combined with interferon-β1a (AVONEX™). In another preferred embodiment, the composition of the invention is memantine combined with interferon-β1a (REBIF™). These compositions are used to treat multiple sclerosis or symptoms arising from multiple sclerosis. Preferably, memantine is administered orally. Preferably, AVONEX™ and REBIF™ are administered subcutaneously or intramuscularly.

In another preferred embodiment, the composition of the invention is a triple combination. Preferably, this triple combination composition of the invention is memantine combined with interferon-β and natalizumab (ANTEGREN™).

The present invention provides a more effective method of treatment for multiple sclerosis, and pharmaceutical compositions for treating MS (or other demyelinating condition) which may be used in such methods. In an embodiment, the invention relates to methods for treating a subject having a demyelinating condition, such as MS, through the administration of a composition containing one or more uncompetitive NMDA receptor channel antagonists in combination with a multiple sclerosis agents or treatments, such as interferon-βs, glatiramer, natalizumab, or daclizumab.

In one embodiment, methods of treating a demyelinating condition, such as multiple sclerosis, are disclosed, wherein a uncompetitive NMDA receptor channel antagonist and a multiple sclerosis agent are administered to a subject having a demyelinating condition such as multiple sclerosis, such that the demyelinating condition is treated or at least partially alleviated. The uncompetitive NMDA receptor channel antagonist and multiple sclerosis agent are administered as part of a pharmaceutical composition. In another embodiment, a patient is diagnosed, e.g., to determine if treatment is necessary, whereupon a composition in accordance with the invention is administered to treat the patient. The amount of uncompetitive NMDA receptor channel antagonist and multiple sclerosis agent is typically effective to reduce symptoms and to enable an observation of a reduction in symptoms.

In addition to the specific combinations disclosed herein, combinations made of a first NMDAr antagonist and the second agent (MS agent as described herein) may be identified by testing the ability of a test combination of a selected NMDAr antagonist and one or more second agents that reduces symptoms of MS or demyelinating conditions. Preferred combinations are those in which a lower therapeutically effective amount of the NMDA receptor antagonist and/or the second agent (e.g., MS agent(s)) is present relative to the same amount of the NMDA receptor antagonist and/or the second agent required to obtain the same effect when each agent is tested separately.

The amounts and ratios of the NMDA receptor antagonist and the second agent are conveniently varied to maximize the therapeutic benefit and minimize the toxic or safety concerns. The NMDA receptor antagonist may range between 20% and 200% of its normal effective dose and the second agent may range between 20% to 200% of its normal effective dose. The precise ratio may vary according to the condition being treated. In one example, the amount of memantine ranges between 2.5 and 40 mg per day and the amount of tizanidine hydrochloride ranges between 5 and 75 mg/day.

In addition to the specific combinations disclosed herein, combinations made of an NMDA receptor antagonist such as an aminoadamantane compound and a second agent which is a MS agent may be identified by testing the ability of a test combination to reduce the symptoms of MS or any demyelinating condition described in the instant specification.

For a specified range a physician or other appropriate health professional will typically determine the best dosage for a given patient, according to his sex, age, weight, pathological state, and other parameters. In some cases, it may be necessary to use dosages outside of the ranges stated in pharmaceutical packaging insert to treat a subject. Those cases will be apparent to the prescribing physician.

In some embodiments, the combinations of the invention achieve therapeutic levels while minimizing debilitating side-effects that are usually associated with immediate release formulations. Furthermore, as a result of the delay in the time to obtain peak plasma level and the potentially extended period of time at the therapeutically effective plasma level, the dosage frequency may be reduced to, for example, once or twice daily dosage, thereby improving patient compliance and adherence.

Accordingly, the combination of the invention allows the NMDA receptor antagonist and the second agent to be administered in a combination that improves efficacy and avoids undesirable side effects of both drugs. For example, side effects including psychosis and cognitive deficits associated with the administration of NMDA receptor antagonists may be lessened in severity and frequency through the use of controlled-release methods that shift the Tmax to longer times, thereby reducing the dC/dT of the drug. Reducing the dC/dT of the drug not only increases Tmax, but also reduces the drug concentration at Tmax and reduces the Cmax/Cmean ratio providing a more constant amount of drug to the subject being treated over a given period of time and reducing adverse events associated with dosing. Also, side effects including myelosuppression, flu-like symptoms, gastrointestinal disorders, dizziness, coughing, retinopathy, thyroiditis, acute pancreatitis, and depression associated with interferons, are lessened in severity and frequency through the use of the combination therapy of the invention.

In certain embodiments, the combinations provide additive effects. Additivity is achieved by combining the active agents without requiring controlled release technologies. In other embodiments, particularly when the pharmacokinetic profiles of the combined active pharmaceutical ingredients are dissimilar, controlled release formulations optimize the pharmacokinetics of the active pharmaceutical agents to reduce the variability of the Cratio over time. Reduction of Cratio variability over a defined time period enables a concerted effect for the agents over that time, maximizing the effectiveness of the combination. The Cratio variability (“Cratio.var”) is defined as the standard deviation of a series of Cratios taken over a given period of time divided by the mean of those Cratios multiplied by 100%. Compositions of a uncompetitive NMDA receptor channel antagonist, e.g., memantine, rimantadine, amantadine, and pharmaceutically acceptable salts and esters thereof; and multiple sclerosis agents such as β-interferons are synergistically effective and are effective in treating a demyelinating disorder such as MS.

Synergy is defined as the interaction of two or more agents so that their combined effect is greater than the sum of their individual effects. For example, if the effect of drug A alone in treating a disease is 25%, and the effect of drug B alone in treating a disease is 25%, but when the two drugs are combined the effect in treating the disease is 75%, the effect of A and B is synergistic.

Additivity is defined as the interaction of two or more agents so that their combined effect is greater than the sum of their individual effects. For example, if the effect of drug A alone in treating a disease is 25%, and the effect of drug B alone in treating a disease is 25%, but when the two drugs are combined the effect in treating the disease is greater than 25%, the effect of A and B is additive.

An improvement in the drug therapeutic regimen can be described as the interaction of two or more agents so that their combined effect reduces the incidence of adverse event (AE) of either or both agents used in co-therapy. This reduction in the incidence of adverse effects can be a result of, e.g., administration of lower dosages of either or both agent used in the co-therapy. For example, if the effect of Drug A alone is 25% and has an adverse event incidence of 45% at labeled dose; and the effect of Drug B alone is 25% and has an adverse event incidence of 30% at labeled dose, but when the two drugs are combined at lower than labeled doses of each, if the overall effect is 35%. and the adverse incidence rate is 20%, there is an improvement in the drug therapeutic regimen.

In one embodiment, methods of treating a demyelinating condition, such as multiple sclerosis, are disclosed, wherein a uncompetitive NMDA receptor channel antagonist and a multiple sclerosis agent are administered to a subject having a demyelinating condition such as multiple sclerosis, such that the demyelinating condition is treated or at least partially alleviated. The uncompetitive NMDA receptor channel antagonist and multiple sclerosis agent may be administered as part of a pharmaceutical composition, or as part of a combination therapy. In another embodiment, a patient is diagnosed, e.g., to determine if treatment is necessary, whereupon a combination therapy in accordance with the invention is administered to treat the patient. The amount of uncompetitive NMDA receptor channel antagonist and multiple sclerosis agent is typically effective to reduce symptoms and to enable an observation of a reduction in symptoms.

Combination therapies of a uncompetitive NMDA receptor channel antagonist, e.g., memantine, rimantadine, amantadine, and pharmaceutically acceptable salts and esters thereof; and multiple sclerosis agents such as β-interferons are synergistically effective and are effective in treating a demyelinating disorder such as MS.

Dosages Taken Together

The uncompetitive NMDA receptor channel antagonist used in combination therapies of the invention are administered at a dosage of generally, for memantine from about 5 to about 20 mg/day, for amantadine from about 50 to about 200 mg/day, and for rimantadine from about 50 to about 200 mg/day. Memantine is particularly preferred.

When interferon-β1a (i.e., AVONEX™) is used, it is administered at a dosage of about 7.5 to about 30 μg preferably intramuscularly, once a week. When interferon-β1a (i.e., REBIF™) is used, it is administered at a dosage of about 11 μg to about 44 μg, preferably subcutaneously and preferably three times a week. When interferon-β1b (e.g., BETASERON™) is used, it is administered at a dosage of about 50 μg to about 250 μg, preferably subcutaneously and preferably every other day. When glatiramer acetate (e.g., COPAXONE™) is used, it is administered at a dosage of about 5 mg to about 20 mg, e.g., and is preferably administered subcutaneously, preferably daily. When natalizumab (ANTEGREN™) is used, it is administered at a dosage of about 1.5 mg/kg to 6 mg/kg by intravenous infusion, preferably once every four weeks. When mitoxanthrone (NOVANTRONE™) is used for reducing neurologic disability and/or the frequency of clinical relapses in patients with secondary (chronic) progressive, progressive relapsing, or worsening relapsing remitting multiple sclerosis, the recommended dosage of NOVANTRONE is about 3 to about 12 mg/m² given as a short (approximately 5 to 15 minutes) intravenous infusion every 3 months. When daclizumab (ZENAPAX™) is used, it is administered at a dosage of about 0.25 to 1 mg/kg (intravenous) every 14 days, for a total of 5 doses.

Modes of Administration

The combination of the invention may be administered in either a local or systemic manner or in a depot or sustained release fashion. The two agents may be delivered in an oral, transdermal or intranasal formulation. In a preferred embodiment, the NMDA receptor antagonist, the second agent of the combination (MS agent as described herein), or both agents may be formulated to provide controlled, extended release or immediate release as described herein. For example, a pharmaceutical composition that provides controlled release of the NMDA receptor antagonist, the second agent, or both may be prepared by combining the desired agent or agents with one or more additional ingredients that, when administered to a subject, causes the respective agent or agents to be released at a targeted rate for a specified period of time. The two agents are preferably administered in a manner that provides the desired effect from the first and second agents in the combination. Optionally, the first and second agents are admixed into a single formulation before they are introduced into a subject. The combination may be conveniently sub-divided in unit doses containing appropriate quantities of the first and second agents. The unit dosage form may be, for example, a capsule or tablet itself or it can be an appropriate number of such compositions in package form. The quantity of the active ingredients in the unit dosage forms may be varied or adjusted according to the particular need of the condition being treated.

Alternatively, the NMDA receptor antagonist and the second agent of the combination may not be mixed until after they are introduced into the subject. Thus, the term “combination” encompasses embodiments where the NMDA receptor antagonist and the second agent are provided in separate formulations and are administered sequentially. For example, the NMDA receptor antagonist and the second agent may be administered to the subject separately within 2 days, 1 day, 18 hours, 12 hours, one hour, a half hour, 15 minutes, or less of each other. Each agent may be provided in multiple, single capsules or tablets that are administered separately to the subject. Alternatively, the NMDA receptor antagonist and the second agent are separated from each other in a pharmaceutical composition such that they are not mixed until after the pharmaceutical composition has been introduced into the subject. The mixing may occur just prior to administration to the subject or well in advance of administering the combination to the subject.

Schedule of Administration

As noted above, combination therapies of a uncompetitive NMDA receptor channel antagonist and a multiple sclerosis agent are part of the invention. The combination therapies of the invention are administered in any suitable fashion to obtain the desired treatment of a demyelinating disease (e.g., multiple sclerosis) in the patient. One way in which this is achieved is to prescribe a regimen of uncompetitive NMDA receptor channel antagonist so as to “pre-treat” the patient to obtain the effects of the uncompetitive NMDA receptor channel antagonist (e.g. a slowing of disease progression and neuroprotection), then follow that up with the multiple sclerosis agent as part of a specific treatment regimen, e.g., a standard administration of interferon-β1a, e.g., intramuscularly or subcutaneously, to provide the benefit of the co-action of the therapeutic agents. Combination therapies of the invention include this sequential administration, as well as administration of these therapeutic agents, or at least two of the therapeutic agents, in a substantially simultaneous manner. Substantially simultaneous administration can be accomplished, for example, by administering to the subject a single capsule (e.g., a single composition) or injection having a fixed ratio of a uncompetitive NMDA receptor channel antagonist and, e.g., a β-interferon, or in multiple, single capsules or injections. The components of the combination therapies, as noted above, can be administered by the same route or by different routes. For example, an uncompetitive NMDA receptor channel antagonist is administered by orally, while the multiple sclerosis agents is administered intramuscularly or subcutaneously; or all therapeutic agents may be administered orally or all therapeutic agents may be administered by intravenous injection. The sequence in which the therapeutic agents are administered is not believed to be critical.

Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also can embrace the administration of the therapeutic agents as described above in further combination with other biologically active ingredients and non-drug therapies (e.g., surgery or radiation treatment) or other treatment modalities like interventional treatment regiments. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporarily removed from the administration of the therapeutic agents, perhaps by days or even weeks.

Thus, the compounds of the invention and the other pharmacologically active agent may be administered to a patient simultaneously, sequentially or in combination. If administered sequentially, the time between administrations generally varies from 0.1 to about 48 hours. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

A combination therapy for a demyelinating condition includes memantine and β-interferon. In another embodiment, a combination therapy for a demyelinating condition includes rimantadine and β-interferon. In an embodiment, a combination therapy for a demyelinating condition includes amantadine and β-interferon.

In another embodiment, a combination therapy for a demyelinating condition includes memantine and glatiramer. In one embodiment, a combination therapy for a demyelinating condition includes rimantadine and glatiramer. In an embodiment, a combination therapy for a demyelinating condition includes amantadine and glatiramer.

In yet another embodiment, a combination therapy for a demyelinating condition includes memantine and natalizumab. In another embodiment, a combination therapy for a demyelinating condition includes rimantadine and natalizumab. In yet another embodiment, a combination therapy for a demyelinating condition includes amantadine and natalizumab.

In yet another embodiment, a combination therapy for a demyelinating condition includes memantine and daclizumab. In another embodiment, a combination therapy for a demyelinating condition includes rimantadine and daclizumab. In yet another embodiment, a combination therapy for a demyelinating condition includes amantadine and daclizumab.

In yet another embodiment, a combination therapy for a demyelinating condition includes memantine and mitoxanthrone. In another embodiment, a combination therapy for a demyelinating condition includes rimantadine and mitoxanthrone. In yet another embodiment, a combination therapy for a demyelinating condition includes amantadine and mitoxanthrone. In a preferred embodiment, the demyelinating condition is MS.

The present invention provides a more effective method of treatment for multiple sclerosis, and pharmaceutical compositions for treating MS (or other demyelinating condition) which may be used in such methods. In an embodiment, the invention relates to methods for treating multiple sclerosis through the administration of one or more uncompetitive NMDA receptor channel antagonists in combination with a multiple sclerosis agents or treatments, such as interferon-βs, glatiramer, natalizumab, mitoxanthrone or daclizumab.

“Combination therapy” (or “co-therapy”) includes the administration of a compound of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. In one embodiment, the co-action of the therapeutic agents is additive. In another embodiment, the co-action of the therapeutic agents is synergistic. In another embodiment, the co-action of the therapeutic agents improves the therapeutic regimen of one or both of the agents.

The invention further relates to kits for treating patients having a demyelinating condition, such as multiple sclerosis, comprising a therapeutically effective dose of an agent for treating or at least partially alleviating the symptoms of the condition (e.g., β-interferons, glatiramer acetate, natalizumab, mitoxanthrone or daclizumab), and a uncompetitive NMDA receptor channel antagonist, either in the same or separate packaging, and instructions for its use.

For example, in one embodiment the demyelinating condition is MS, and the kit includes therapeutic doses of memantine and β-interferon, for treating a patient in need of MS treatment, and instructions for use. In another embodiment, a kit includes therapeutic doses of rimantadine and β-interferon, for treating a patient in need of MS treatment, and instructions for use. In an embodiment, a kit includes therapeutic doses of amantadine and β-interferon, for treating a patient in need of MS treatment, and instructions for use. In another embodiment, a kit includes therapeutic doses of memantine and glatiramer, for treating a patient in need of MS treatment, and instructions for use. In one embodiment, a kit includes therapeutic doses of rimantadine and glatiramer, for treating a patient in need of MS treatment, and instructions for use. In an embodiment, a kit includes therapeutic doses of amantadine and glatiramer, for treating a patient in need of MS treatment, and instructions for use. In yet another embodiment, a kit includes therapeutic doses of memantine and natalizumab, for treating a patient in need of MS treatment, and instructions for use. In one embodiment, a kit includes therapeutic doses of rimantadine and natalizumab, for treating a patient in need of MS treatment, and instructions for use. In yet another embodiment, a kit includes therapeutic doses of amantadine and natalizumab, for treating a patient in need of MS treatment, and instructions for use. In yet another embodiment, a kit includes therapeutic doses of memantine and daclizumab, for treating a patient in need of MS treatment, and instructions for use. In one embodiment, a kit includes therapeutic doses of rimantadine and daclizumab, for treating a patient in need of MS treatment, and instructions for use. In yet another embodiment, a kit includes therapeutic doses of amantadine and daclizumab, for treating a patient in need of MS treatment, and instructions for use. In another embodiment, a kit includes therapeutic doses of memantine and mitoxanthrone, for treating a patient in need of MS treatment, and instructions for use. In one embodiment, a kit includes therapeutic doses of rimantadine and mitoxanthrone, for treating a patient in need of MS treatment, and instructions for use. In an embodiment, a kit includes therapeutic doses of amantadine and mitoxanthrone, for treating a patient in need of MS treatment, and instructions for use.

The present invention is suitable for the reduction of multiple sclerosis symptoms. These multiple sclerosis symptoms include perturbations of pyramidal functions, e.g., development of paraparesis, hemiparesis, monoparesis and quadriparesis and the development of monoplegia, paraplegia, quadriplegia, and hemiplegia. The symptoms of multiple sclerosis also include perturbations in cerebellar functions. These perturbations include the development of ataxia, including truncal and limb ataxia. “Paralytic symptoms of multiple sclerosis” includes these perturbations in pyramidal and cerebellar functions.

The symptoms of multiple sclerosis also include changes in brain stem functions including development of nystagmus and extraocular weakness along with dysarthria. Further symptoms include loss of sensory function including decrease in touch or position sense and loss of sensation in limbs. Perturbations in bowel and bladder function, including hesitancy, urgency, retention of bowel or bladder or incontinence, can also occur. Visual functions such as scotoma development are also affected by multiple sclerosis. Cerebral function degeneration, including a decrease in mentation and the development of dementia, is also a symptom.

To evaluate whether a patient is benefiting from the (treatment), one would examine the patient's symptoms in a quantitative way, by decrease in the frequency of relapses, or increase in the time to sustained progression, or improvement in the magnetic resonance imaging (MRI) behavior in frequent, serial MRI studies and compare the patient's status measurement before and after treatment. In a successful treatment, the patient status will have improved (i.e., the Extended Disability Status Scale (EDSS), described in the Examples below, measurement number or frequency of relapses will have decreased, or the time to sustained progression will have increased, or the MRI scans will show less pathology).

As for every drug, the dosage is an important part of the success of the treatment and the health of the patient. In every case, in the specified range, the physician has to determine the best dosage for a given patient, according to gender, age, weight, height, pathological state and other parameters.

The pharmaceutical compositions of the present invention contain a therapeutically effective amount of the active agents. The amount of the compound will depend on the patient being treated. The patient's weight, severity of illness, manner of administration and judgment of the prescribing physician should be taken into account in deciding the proper amount. The determination of a therapeutically effective amount of an uncompetitive NMDA receptor channel antagonist or multiple sclerosis agent is well within the capabilities of one with skill in the art.

In some cases, it may be necessary to use dosages outside of the ranges stated in pharmaceutical packaging insert to treat a patient. Those cases will be apparent to the prescribing physician. Where it is necessary, a physician will also know how and when to interrupt, adjust or terminate treatment in conjunction with a response of a particular patient.

Formulation (Separately or Together) and Administration

The compounds of the present invention are administered separately or co-formulated in a suitable co-formulated dosage form. Compounds, including those used in combination therapies are administered to a patient in the form of a pharmaceutically acceptable salt or in a pharmaceutical composition. A compound that is administered in a pharmaceutical composition is mixed with a suitable carrier or excipient such that a therapeutically effective amount is present in the composition. The term “therapeutically effective amount” refers to an amount of the compound that is necessary to achieve a desired endpoint (e.g., decreasing symptoms associated with demyelination).

A variety of preparations can be used to formulate pharmaceutical compositions containing the uncompetitive NMDA receptor channel antagonist and multiple sclerosis agents. Techniques for formulation and administration may be found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants and aerosols are examples of such formulations. The formulations can be administered in either a local or systemic manner or in a depot or sustained release fashion. Administration of the composition can be performed in a variety of ways. The compositions and combination therapies of the invention may be administered in combination with a variety of pharmaceutical excipients, including stabilizing agents, carriers and/or encapsulation formulations as described herein.

The preparation of pharmaceutical or pharmacological compositions will be known to those of skill in the art in light of the present disclosure. Typically, such compositions may be prepared as injectables, either as liquid solutions suspensions or gels; solid forms suitable for solution in, or suspension in, liquid prior to injection; as tablets, pills or other solids for oral administration; as time release capsules; or in any other form currently used, including creams, lotions, mouthwashes, inhalants and the like.

Alternatively, the compositions of the present invention may be administered transdermally via a number of strategies, including those described in U.S. Pat. Nos. 5,186,938, 6,183,770, 4,861,800 and WO 89/09051. Providing the drugs of the combination in the form of patches is particularly useful given that these agents have relatively high skin fluxes.

Pharmaceutical compositions containing the NMDA receptor antagonist and/or second agent of the combination may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage may be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

In some embodiments, for example, the composition may be delivered intranasally to the cribriform plate rather than by inhalation to enable transfer of the active agents through the olfactory passages into the CNS and reducing the systemic administration. Devices used for this route of administration are included in U.S. Pat. No. 6,715,485. Compositions delivered via this route may enable increased CNS dosing or reduced total body burden reducing systemic toxicity risks associated with certain drugs.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The combination may optionally be formulated for delivery in a vessel that provides for continuous long-term delivery, e.g., for delivery up to 30 days, 60 days, 90 days, 180 days, or one year. For example the vessel can be provided in a biocompatible material such as titanium. Long-term delivery formulations are particularly useful in subjects with chronic conditions, for assuring improved patient compliance, and for enhancing the stability of the combinations. Formulations for continuous long-term delivery are provided in, e.g., U.S. Pat. Nos. 6,797,283; 6,764,697; 6,635,268, and 6,648,083.

The NMDA receptor antagonist, the second agent of the invention, or both agents may be provided in a controlled, extended release form. In one example, at least 50%, 90%, 95%, 96%, 97%, 98%, 99%, or even in excess of 99% of the NMDA receptor antagonist is provided in an extended release dosage form. A release profile, i.e., the extent of release of the NMDA receptor antagonist or the second agent over a desired time, may be conveniently determined for a given time by calculating the Cₘₐₓ/Cₘₑₐₙ for a desired time range to achieve a given acute or chronic steady state serum concentration profile. Thus, upon the administration to a subject (e.g., a mammal such as a human), the NMDA receptor antagonist has a Cmax/Cmean of approximately 2.5, 2, 1.5, or 1.0 approximately 1, 1.5, 2 hours to at least 6, 8, 9, 12, 18, 21, or 24 hours following such administration. If desired, the release of the NMDA receptor antagonist may be monophasic or multiphasic (e.g., biphasic). Moreover, the second agent may be formulated as an extended release composition, having a Cₘₐₓ/Cₘₑₐₙ of approximately 2.5, 2, 1.5, or 1.0, approximately 1, 1.5, 2 hours to at least 6, 8, 9, 12, 18, 21, 24 hours following administration to a subject. One of ordinary skill in the art can prepare combinations with a desired release profile using the NMDA receptor antagonists and the second agent and formulation methods known in the art or described below.

As shown in Tables 1 and 2, the pharmacokinetic half-lives of the drugs of both classes varies from about 1.5 hours to 70 hours. Thus, suitable formulations may be conveniently selected to achieve nearly constant concentration profiles over an extended period (preferably from 8 to 24 hours) thereby maintaining both agents in a constant ratio and concentration for optimal therapeutic benefits for both acute and chronic administration. Preferred Cratio,var values may be less than about 30%, 50%, 75%, 90% of those for IR administration of the same active pharmaceutical ingredients over the first 4, 6, 8, 12 hours after administration. Preferred Cratio,var values are less than about 100%, 70%, 50%, 30%, 20%, 10%.

Formulations that deliver this constant, measurable profile also allow one to achieve a monotonic ascent from an acute ratio to a desired chronic ratio for drugs with widely varying elimination half-lives. Compositions of this type and methods of treating patients with these compositions are embodiments of the invention. Numerous ways exist for achieving the desired release profiles, as exemplified below.

Suitable methods for preparing combinations in which the first agent, second agent, or both agents are provided in extended release-formulations include those described in U.S. Pat. No. 4,606,909 (hereby incorporated by reference). This reference describes a controlled release multiple unit formulation in which a multiplicity of individually coated or microencapsulated units are made available upon disintegration of the formulation (e.g., pill or tablet) in the stomach of the animal (see, for example, column 3, line 26 through column 5, line 10 and column 6, line 29 through column 9, line 16). Each of these individually coated or microencapsulated units contains cross-sectionally substantially homogenous cores containing particles of a sparingly soluble active substance, the cores being coated with a coating that is substantially resistant to gastric conditions but which is erodable under the conditions prevailing in the small intestine.

The combination may alternatively be formulated using the methods disclosed in U.S. Pat. No. 4,769,027, for example. Accordingly, extended release formulations involve pills of pharmaceutically acceptable material (e.g., sugar/starch, salts, and waxes) may be coated with a water permeable polymeric matrix containing an NMDA receptor antagonist and next overcoated with a water-permeable film containing dispersed within it a water soluble particulate pore forming material.

One or both agents of the combination may additionally be prepared as described in U.S. Pat. No. 4,897,268, involving a biocompatible, biodegradable microcapsule delivery system. Thus, the NMDA receptor antagonist may be formulated as a composition containing a blend of free-flowing spherical particles obtained by individually microencapsulating quantities of memantine, for example, in different copolymer excipients which biodegrade at different rates, therefore releasing memantine into the circulation at a predetermined rates. A quantity of these particles may be of such a copolymer excipient that the core active ingredient is released quickly after administration, and thereby delivers the active ingredient for an initial period. A second quantity of the particles is of such type excipient that delivery of the encapsulated ingredient begins as the first quantity's delivery begins to decline. A third quantity of ingredient may be encapsulated with a still different excipient which results in delivery beginning as the delivery of the second quantity beings to decline. The rate of delivery may be altered, for example, by varying the lactide/glycolide ratio in a poly(D,L-lactide-co-glycolide) encapsulation. Other polymers that may be used include polyacetal polymers, polyorthoesters, polyesteramides, polycaprolactone and copolymers thereof, polycarbonates, polyhydroxybuterate and copolymers thereof, polymaleamides, copolyaxalates and polysaccharides.

Alternatively, the combination may be prepared as described in U.S. Pat. No. 5,395,626 features a multilayered controlled release pharmaceutical dosage form. The dosage form contains a plurality of coated particles wherein each has multiple layers about a core containing an NMDA receptor antagonist and/or the second agent whereby the drug containing core and at least one other layer of drug active is overcoated with a controlled release barrier layer therefore providing at least two controlled releasing layers of a water soluble drug from the multilayered coated particle.

In some embodiments, the first agent and second agent of the combination described herein are provided within a single or separate pharmaceutical compositions. “Pharmaceutically or Pharmacologically Acceptable” includes molecular entities and compositions that do not produce an adverse, allergic or other untoward reaction when administered to an animal, or a human, as appropriate. “Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. “Pharmaceutically Acceptable Salts” include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The preparation of pharmaceutical or pharmacological compositions are known to those of skill in the art in light of the present disclosure. General techniques for formulation and administration are found in “Remington: The Science and Practice of Pharmacy, Twentieth Edition,” Lippincott Williams & Wilkins, Philadelphia, Pa. Tablets, capsules, pills, powders, granules, dragées, gels, slurries, ointments, solutions suppositories, injections, inhalants and aerosols are examples of such formulations.

By way of example, extended release oral formulation can be prepared using additional methods known in the art. For example, a suitable extended release form of the either active pharmaceutical ingredient or both may be a matrix tablet composition. Suitable matrix forming materials include, for example, waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm oil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. Optionally, the finished tablet may be coated or uncoated.

The coating composition typically contains an insoluble matrix polymer (approximately 15-85% by weight of the coating composition) and a water soluble material (e.g., approximately 15-85% by weight of the coating composition). Optionally an enteric polymer (approximately 1 to 99% by weight of the coating composition) may be used or included. Suitable water soluble materials include polymers such as polyethylene glycol, hydroxypropyl cellulose, hydroxypropyl methyl cellulose, polyvinylpyrrolidone, polyvinyl alcohol, and monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups.

The coating composition may be plasticised according to the properties of the coating blend such as the glass transition temperature of the main agent or mixture of agents or the solvent used for applying the coating compositions. Suitable plasticisers may be added from 0 to 50% by weight of the coating composition and include, for example, diethyl phthalate, citrate esters, polyethylene glycol, glycerol, acetylated glycerides, acetylated citrate esters, dibutylsebacate, and castor oil. If desired, the coating composition may include a filler. The amount of the filler may be 1% to approximately 99% by weight based on the total weight of the coating composition and may be an insoluble material such as silicon dioxide, titanium dioxide, talc, kaolin, alumina, starch, powdered cellulose, MCC, or polacrilin potassium.

The coating composition may be applied as a solution or latex in organic solvents or aqueous solvents or mixtures thereof. If solutions are applied, the solvent may be present in amounts from approximate by 25-99% by weight based on the total weight of dissolved solids. Suitable solvents are water, lower alcohol, lower chlorinated hydrocarbons, ketones, or mixtures thereof. If latexes are applied, the solvent is present in amounts from approximately 25-97% by weight based on the quantity of polymeric material in the latex. The solvent may be predominantly water.

The pharmaceutical composition described herein may also include a carrier such as a solvent, dispersion media, coatings, antibacterial and antifungal agents, isotonic and absorption delaying agents. The use of such media and agents for pharmaceutically active substances is well known in the art. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition may also contain liquids, such as water, saline, glycerol, and ethanol, as well as substances such as wetting agents, emulsifying agents, or pH buffering agents. Liposomes, such as those described in U.S. Pat. Nos. 5,422,120, WO 95/13796, WO 91/14445, or EP 524,968 B1, may also be used as a carrier.

Additional methods for making controlled release formulations are described in, e.g., U.S. Pat. Nos. 5,422,123, 5,601,845, 5,912,013, and 6,194,000, all of which are hereby incorporated by reference.

Preparation for delivery in a transdermal patch can be performed using methods also known in the art, including those described generally in, e.g., U.S. Pat. Nos. 5,186,938 and 6,183,770, 4,861,800, and 4,284,444. A patch is a particularly useful embodiment in cases where the therapeutic agent has a short half-life. Patches can be made to control the release of skin-permeable active ingredients over a 12 hour, 24 hour, 3 day, and 7 day period. In one example, a 2-fold daily excess of an NMDA receptor antagonist is placed in a non-volatile fluid along with the second agent. Given the amount of the agents employed herein, a preferred release will be from 12 to 72 hours.

Transdermal preparations of this form will contain from 1% to 50% active ingredients. The compositions of the invention are provided in the form of a viscous, non-volatile liquid. Preferably, both members of the combination will have a skin penetration rate of at least 10⁻⁹ mole/cm²/hour. At least 5% of the active material will flux through the skin within a 24 hour period. The penetration through skin of specific formulations may be measures by standard methods in the art (for example, Franz et al., J. Invest. Derm. 64:194-195 (1975)).

In some embodiments, the composition may be delivered intranasally to the brain rather than by inhalation to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs.

Preparation of a Pharmaceutical Composition for Delivery in a Subdermally implantable device can be performed using methods known in the art, such as those described in, e.g., U.S. Pat. Nos. 3,992,518; 5,660,848; and 5,756,115.

For human administration, preparations should meet sterility, pyrogenicity, general safety and purity standards as required by the FDA.

Administration of compounds alone or in combination therapies may be, e.g., subcutaneous, intramuscular or intravenous injection, or any other suitable route of administration. A particularly convenient frequency for the administration of the compounds of the invention is once a day.

Upon formulation, therapeutics will be administered in a manner compatible with the dosage formulation, and in such amount as is pharmacologically effective. The formulations are easily administered in a variety of dosage forms, such as the injectable solutions described, but drug release capsules and the like can also be employed. In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

A carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Suitable preservatives for use in solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The compounds and combination therapies of the invention can be formulated by dissolving, suspending or emulsifying in an aqueous or nonaqueous solvent. Vegetable (e.g., sesame oil, peanut oil) or similar oils, synthetic aliphatic-acid glycerides, esters of higher aliphatic acids and propylene glycol are examples of nonaqueous solvents. Aqueous solutions such as Hank's solution, Ringer's solution or physiological saline buffer can also be used. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for subcutaneous or intramuscular injection is also contemplated. In this regard, the use of DMSO as solvent is preferred as this will result in extremely rapid penetration, delivering high concentrations of the active compound(s) or agent(s) to a small area.

Where one or both active ingredients of the combination therapy is given orally, it can be formulated through combination with pharmaceutically acceptable carriers that are well known in the art. The carriers enable the compound to be formulated, for example, as a tablet, pill capsule, solution, suspension, sustained release formulation; powder, liquid or gel for oral ingestion by the patient. Oral use formulations can be obtained in a variety of ways, including mixing the compound with a solid excipient, optionally grinding the resulting mixture, adding suitable auxiliaries and processing the granule mixture. The following list includes examples of excipients that can be used in an oral formulation: sugars such as lactose, sucrose, mannitol or sorbitol; cellulose preparations such as maize starch, wheat starch, potato starch, gelatin, gum tragacanth, methyl cellulose, hydroxypropylmethylcellulose, sodium carboxymethylcellulose and polyvinylpyrrolidone (PVP). Oral formulations include such normally employed excipients as, for example, pharmaceutical grades of mannitol, lactose, starch, magnesium stearate, sodium saccharine, cellulose, magnesium carbonate and the like.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The subject treated by the methods of the invention is a mammal, more preferably a human. The following properties or applications of these methods will essentially be described for humans although they may also be applied to non-human mammals, e.g., apes, monkeys, dogs, mice, etc. The invention therefore can also be used in a veterinarian context.

The pharmaceutical compositions of the invention are used to demyelinating conditions. One demyelinating condition that is treated by the pharmaceutical compositions of the invention is multiple sclerosis. Also treated by the pharmaceutical compositions of the invention are symptoms arising from multiple sclerosis, including fatigue, pain and tingling in the arms and legs; localized and generalized numbness, muscle spasm and weakness; bowel and bladder dysfunction; and difficulty with balance when walking or standing. Also treatable by the pharmaceutical compositions of the invention are other demyelinating disorders, such as progressive multifocal leukoencephalopathy (PML) disseminated necrotizing leukoencephalopathy (DNL), acute disseminated encephalomyelitis, Schilder disease, central pontine myelinolysis (CPM), radiation necrosis and Binswanger disease (SAE).

These demyelinating disorders, especially multiple sclerosis, are often characterized by unpredictable attacks where the clinical symptoms become worse (exacerbation) which are separated by periods of remission where the symptoms stabilize or diminish. The pharmaceutical compositions of the invention act by diminishing the periods of exacerbation and extending the periods of remission. In one embodiment, the periods of exacerbation are stopped completely.

In one embodiment, the uncompetitive NMDA receptor channel antagonists of the invention are co-administered with interferon-β. The combination is contemplated to ameliorate the T-cell mediated destruction of myelin basic protein and the B-cell mediated destruction of oligodendroglial cells. The combination is also effective in treating symptoms associated with MS like fatigue.

In one embodiment the combination therapy (e.g. uncompetitive NMDA receptor channel antagonist such as memantine, and interferon-beta) compositions disclosed herein can also be formulated as liposomes. Liposomes containing the compositions of the invention are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Compositions of the present invention can be conjugated to the liposomes as described in Martin et al., J. Biol. Chem., 257: 286-288 (1982) via a disulfide-interchange reaction.

If desired, the agents may be provided in a kit. The kit can additionally include instructions for using the kit. In some embodiments, the kit includes, in one or more containers the NMDA receptor antagonist and, separately, in one or more containers, the second agent described herein (e.g., MS agent). In other embodiments, the kit provides a combination with the NMDA receptor antagonist and the second agent mixed in one or more containers.

Indications Suitable for Treatment with the Combination

Any subject experiencing or at risk of experiencing a demyelating condition such as MS may be treated as described herein. Additional conditions that may be treated using the combinations described herein include acute pain (e.g., post operative acute pain, low back pain, post-herpetic neuralgia, trigeminal neuralgia, spinal cord injury pain, carpal tunnel syndrome, cancer chemotherapy, phantom limb, ischemic pain, and pain due to burns), chronic pain (e.g., musculoskeletal pain, cancer pain, arthritis (including rheumatoid arthritis and osteoarthritis), pain resulting from sports injuries, back pain (such as low back pain), menstrual pain, gastrointestinal or urethral cramps, skin wounds or burns, and cancer pain.

Post operative acute pain and musculoskeletal chronic pain symptoms include any of the following: paraesthesias or dysaesthesias such as burning sensation, sharp pain, lightning pain, lancinating pain, paroxysmal pain, dull, achy pain, pins and needles sensation, referred pain, areas of the skin with diminished sensation, areas of heightened sensation, areas of abnormal sensation, reddened skin, skin hairs standing up, loss of hair, ulceration of skin, thinning of skin

Moreover, any CNS-related disorder, such as dementias (e.g., Alzheimer's disease, Parkinson's disease, Picks disease, fronto-temporal dementia, vascular dementia, normal pressure hydrocephalus, HD, and MCl), neuro-related conditions, dementia-related conditions, such as epilepsy, seizure disorders, acute pain, chronic pain, chronic neuropathic pain may be treated using the combinations and methods described herein. Epileptic conditions include complex partial, simple partial, partials with secondary generalization, generalized—including absence, grand mal (tonic clonic), tonic, atonic, myoclonic, neonatal, and infantile spasms. Additional specific epilepsy syndromes are juvenile myoclonic epilepsy, Lennox-Gastaut, mesial temporal lobe epilepsy, nocturnal frontal lobe epilepsy, progressive epilepsy with mental retardation, and progressive myoclonic epilepsy. The combinations of the invention are also useful for the treatment and prevention of pain caused by disorders including cerebrovascular disease, motor neuron diseases (e.g., ALS, Spinal motor atrophies, Tay-Sach's, Sandoff disease, familial spastic paraplegia), neurodegenerative diseases (e.g., familial Alzheimer's disease, prion-related diseases, cerebellar ataxia, Friedrich's ataxia, SCA, Wilson's disease, RP, ALS, Adrenoleukodystrophy, Menke's Sx, cerebral autosomal dominant arteriopathy with subcortical infarcts (CADASIL); spinal muscular atrophy, familial ALS, muscular dystrophies, Charcot Marie Tooth diseases, neurofibromatosis, von-Hippel Lindau, Fragile X, spastic paraplesia, psychiatric disorders (e.g., panic syndrome, general anxiety disorder, phobic syndromes of all types, mania, manic depressive illness, hypomania, unipolar depression, depression, stress disorders, PTSD, somatoform disorders, personality disorders, psychosis, and schizophrenia), and drug dependence (e.g., alcohol, psychostimulants (eg, crack, cocaine, speed, meth), opioids, and nicotine), Tuberous sclerosis, and Wardenburg syndrome), strokes (e.g, thrombotic, embolic, thromboembolic, hemmorhagic, venoconstrictive, and venous), movement disorders (e.g., PD, dystonias, benign essential tremor, tardive dystonia, tardive dyskinesia, and Tourette's syndrome), ataxic syndromes, disorders of the sympathetic nervous system (e.g., Shy Drager, Olivopontoicerebellar degeneration, striatonigral degenration, PD, HD, Guillain Barre, causalgia, complex regional pain syndrome types I and II, diabetic neuropathy, and alcoholic neuropathy), Cranial nerve disorders (e.g., Trigeminal neuropathy, trigeminal neuralgia, Menier's syndrome, glossopharangela neuralgia, dysphagia, dysphonia, and cranial nerve palsies), myelopethies, traumatic brain and spinal cord injury, radiation brain injury, Post-menengitis syndrome, prion diseases, myelities, radiculitis, neuropathies (e.g., Guillain-Barre, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy), axonic brain damage, encephalopathies, and chronic fatigue syndrome. Pain associated with any of these conditions may be treated using the methods and compositions described herein. All of the above disorders may be treated with the combinations described herein, whether pain is involved or not.

Immediate release formulations of memantine (e.g., Namenda) are typically administered at low doses (e.g., 5 mg/day) and progressively administered at increasing frequency and dose over time to reach a steady state serum concentration that is therapeutically effective. Namenda, an immediate release formulation of memantine, is first administered to subjects at a dose of 5 mg per day. After a period of time, subjects are administered with this dose twice daily. Subjects are next administered with a 5 mg and 10 mg dosing per day and finally administered with 10 mg Namenda twice daily. Using this dosing regimen, a therapeutically effective steady state serum concentration may be achieved within about thirty days following the onset of therapy. Using a sustained release formulation (22.5 mg) however, a therapeutically effective steady state concentration may be achieved substantially sooner, without using a dose escalating regimen. Such concentration is predicted to be achieved within 13 days of the onset of therapy. Furthermore, the slope during each absorption period for the sustained release formulation is less (i.e. not as steep) as the slope for Namenda. Accordingly, the dC/dt of the sustained release formulation is reduced relative to the immediate release formulation even though the dose administered is larger than for the immediate release formulation. Based on this model, a sustained release formulation of memantine may be administered to a subject in an amount that is approximately the full strength dose (or that effectively reaches a therapeutically effective dose) from the onset of therapy and throughout the duration of treatment. Accordingly, a dose escalation would not be required.

Treatment of a subject with the combination may be monitored using methods known in the art. If desired, treatment can be monitored by determining if the subject shows a decrease, in one or more of the descriptors associated with demyelating conditions, including those described herein. The efficacy of treatment using the combination is preferably evaluated by examining the subject's symptoms in a quantitative way, e.g., by noting a decrease in the frequency of attacks, or an increase in the time for sustained worsening of symptoms. In a successful treatment, the subject's status will have improved (i.e., frequency of relapses will have decreased, or the time to sustained progression will have increased).

The invention is illustrated in the following non-limiting examples.

### EXAMPLES

In these examples, qualified animal models for MS are employed to examine the dose ranges of synergistic interaction of memantine and MS therapies.

### Example 1

### Treatment with Memantine and Interferon-β1 (AVONEX™ REBIF™ and BETASERON™)

### Animal Models and Methods

Animal Models. The current standard model for testing MS drugs is the Chronic relapsing experimental allergic Encephalomyelitis (EAE) mouse model (Wisniewski, et al., Ann Neurology 1: 144-148 (1977), Bolton et al., Int. Arch. Allergy Immunol. 114: 74-80, 1997).

Procedure: Male Biozzi mice, weighing 25-30 g, receive in each flank 0.15 ml of an emulsion containing lyophilised mouse spinal cord, Freund's complete adjuvant and phosphate buffered saline followed 7 days later by reinoculation at an adjacent site. Between 15 and 22 days after the initial inoculation, sensitised animals suffer body weight loss, hind limb weakness and paralysis. The symptoms resolve, over a 7 day period, and mice enter a remission phase followed, approximately 40 days after inoculation, by a relapse and return of neurological deficits. Neurological signs again remit and return approximately 60 days post-inoculation. Symptoms continue to occur in a cyclic pattern. Test substances or vehicle are administered prophylactically or therapeutically during remission and relapse phases respectively. The ability of drugs to suppress the appearance of neurological signs is assessed against untreated and vehicle dosed, EAE-diseased mice.

Brain and spinal tissues can be removed for histological analysis of the extent of inflammatory cell infiltration by light microscopy following haematoxylin and eosin staining of sections.

Treatment. Cohorts are treated in 4 arms with 2-4 dose ranges of each drug and a placebo, at a compensated dose for animal size, metabolism and circulation, or about ⅙ the mg/kg equivalence. Arm 1: saline, Arm 2: memantine; Arm 3: interferon-β1; Arm 4: memantine plus interferon-β1. These arms are repeated at 2 dose ranges of both memantine and interferon-β1 to measure the dose response relationship.

Study Assessment. Animals are assessed for both arresting inflammation, neuronal degeneration, neurocognitive score and neuromuscular decay. Blood and tissue is analyzed for known surrogate markers.

Results. EAE animals taking both memantine and interferon-β1 display a pronounced reduction in inflammation, neuronal apoptosis and improved neuromuscular behavior.

### Example 2

### Treatment with Memantine and Glatiramer Acetate (COPAXONE™)

EAE mouse model is established by procedures as described in Example 1 of the instant specification

Brain and spinal tissues can be removed for histological analysis of the extent of inflammatory cell infiltration by light microscopy following haematoxylin and eosin staining of sections.

Treatment. Cohorts are treated in 4 arms with 2-4 dose ranges of each drug and a placebo, at a compensated dose for animal size, metabolism and circulation, or about ⅙ the mg/kg equivalence. Arm 1: saline, Arm 2: memantine; Arm 3: glatiramer acetate; Arm 4: memantine plus glatiramer acetate. These arms are repeated at 2 dose ranges of both memantine and glatiramer acetate to measure the dose response relationship.

Study Assessment. Animals are assessed for both arresting inflammation, neuronal degeneration, neurocognitive score and neuromuscular decay. Blood and tissue is analyzed for known surrogate markers.

Results: EAE animals taking both memantine and glatiramer acetate display a pronounced reduction in inflammation, neuronal apoptosis and improved neuromuscular behavior.

### Example 3

### Treatment with Memantine and Natalizumab (ANTEGREN™)

EAE mouse model is established by procedures as described in Example 1 of the instant specification Brain and spinal tissues can be removed for histological analysis of the extent of inflammatory cell infiltration by light microscopy following haematoxylin and eosin staining of sections.

Treatment. Cohorts are treated in 4 arms with 2-4 dose ranges of each drug and a placebo, at a compensated dose for animal size, metabolism and circulation, or about ⅙ the mg/kg equivalence. Arm 1: saline, Arm 2: memantine; Arm 3: natalizumab; Arm 4: memantine plus natalizumab. These arms are repeated at 2 dose ranges of both memantine and natalizumab to measure the dose response relationship.

Study Assessment. Animals are assessed for both arresting inflammation, neuronal degeneration, neurocognitive score and neuromuscular decay. Blood and tissue is analyzed for known surrogate markers.

Results: EAE animals taking both memantine and natalizumab display a pronounced reduction in inflammation, neuronal apoptosis and improved neuromuscular behavior.

### Example 4

### Treatment with Memantine and Mitoxanthrone (NOVANTRONE™)

EAE mouse model is established by procedures as described in Example 1 of the instant specification

Brain and spinal tissues can be removed for histological analysis of the extent of inflammatory cell infiltration by light microscopy following haematoxylin and eosin staining of sections.

Treatment. Cohorts are treated in 4 arms with 2-4 dose ranges of each drug and a placebo, at a compensated dose for animal size, metabolism and circulation, or about ⅙ the mg/kg equivalence. Arm 1: saline, Arm 2: memantine; Arm 3: mitoxanthrone; Arm 4: memantine plus mitoxanthrone. These arms are repeated at 2 dose ranges of both memantine and mitoxanthrone to measure the dose response relationship.

Study Assessment. Animals are assessed for both arresting inflammation, neuronal degeneration, neurocognitive score and neuromuscular decay. Blood and tissue is analyzed for known surrogate markers.

Results: EAE animals taking both memantine and mitoxanthrone display a pronounced reduction in inflammation, neuronal apoptosis and improved neuromuscular behavior.

### Example 5

### Treatment with Memantine and Daclizumab (ZENAPAX™)

EAE mouse model is established by procedures as described in Example 1 of the instant specification Brain and spinal tissues can be removed for histological analysis of the extent of inflammatory cell infiltration by light microscopy following haematoxylin and eosin staining of sections.

Treatment. Cohorts are treated in 4 arms with 2-4 dose ranges of each drug and a placebo, at a compensated dose for animal size, metabolism and circulation, or about ⅙ the mg/kg equivalence. Arm 1: saline, Arm 2: memantine; Arm 3: daclizumab; Arm 4: memantine plus daclizumab. These arms are repeated at 2 dose ranges of both memantine and daclizumab to measure the dose response relationship.

Study Assessment. Animals are assessed for both arresting inflammation, neuronal degeneration, neurocognitive score and neuromuscular decay. Blood and tissue is analyzed for known surrogate markers.

Results: EAE animals taking both memantine and daclizumab display a pronounced reduction in inflammation, neuronal apoptosis and improved neuromuscular behavior.

### Example 6

### Treatment with Memantine and/or Interferon-β (AVONEX™) (REBIF™) or (BETASERON™)

In this example, a comparative study of treatment regimens for MS is described.

### Human Patients and Methods

Patients. Patients eligible for this study include IFN-naïve patients, between the ages of 18-55, diagnosed within the past 2 years with relapsing-remitting MS (RR-MS). Such patients will typically have evidence of demyelination on MRI scanning of the brain and have an Extended Disability Status Scale (EDSS) score between 0 and 3.5.

Treatment. Patients are randomized to receive 1 of 6 study arms: Arm 1: memantine 20 mg orally once per day. Arm 2: interferon-β Avonex at 30 μg dose intramuscularly once weekly or Arm 3 Rebif 44 μg dose subcutaneously 3× weekly. Arm 4: memantine at 5-20 mg/day plus Avonex at 7.5-30 μg. Arm 4: Memantine 5-20 mg/day plus Betaseron at 50-250 μg sc every other day. Arm 5 Memantine 5-20 mg/day plus Rebif at 11-44 μg sc three times per week. Arm 6 Placebo. The study lasts a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

### Results

Of the patients chosen for the study, 25 are randomized to receive memantine, 25 are randomized to receive interferon-β1a, 25 are randomized to receive a combination therapy and 25 are randomized to receive a placebo. Patients taking memantine with interferon-β1a exhibit a decrease in the number of relapses and MRI abnormalities compared with patients treated with either memantine or interferon-β1a alone.

### Example 7

### Treatment with Memantine and/or Glatiramer Acetate (COPAXONE™)

In this example, a comparative study of treatment regimens for MS is described.

### Patients and Methods

Patients. Patients eligible for this study include IFN-naïve patients, between the ages of 18-55, diagnosed within the past 2 years with relapsing-remitting MS (RR-MS). Such patients will typically have evidence of demyelination on MRI scanning of the brain and will have an Extended Disability Status Scale (EDSS) score between 0 and 3.5.

Treatment. Patients are randomized to receive 1 of 4 study arms: Arm 1: memantine 20 mg orally once per day. Arm 2: glatiramer acetate 20 mg subcutaneously once per day. Arm 3: memantine 5 to 20 mg/day plus glatiramer acetate 5 to 20 mg/day. Arm 4: Placebo. The study lasts a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive memantine, 25 are randomized to receive glatiramer acetate, 25 are randomized to receive a combination therapy and 25 are randomized to receive a placebo. Patients taking memantine with glatiramer acetate exhibit a decrease in the number of relapses and MRI abnormalities compared with patients treated with either memantine or glatiramer acetate alone.

### Combination Treatments:

The following combination therapies are specifically contemplated:

Patients. Patients are IFN-naïve patients, between the ages of 18-55, diagnosed within the past 2 years with Relapsing-remitting MS (RR-MS). Patients typically have evidence of demyelination on MRI scanning of the brain and will have an Extended Disability Status Scale (EDSS) score between 0 and 3.5.

Treatment. Patients are randomized to receive one of the following study arms:

memantine 5-20 mg oral once per day and interferon-β1a (AVONEX™) 7.5 to 30 μg IM once weekly;
rimantadine 50-200 mg oral once per day and interferon-β1a (AVONEX™) 7.5 to 30 μg IM once weekly;
amantadine 50-200 mg oral once per day and interferon-β1a (AVONEX™) 7.5 to 30 μg IM once weekly;
memantine 5-20 mg oral once per day and glatiramer acetate 5-20 mg SC once per day;
rimantadine 50-200 mg oral once per day and glatiramer acetate 5-20 mg SC once per day;
amantadine 50-200 mg oral once per day and glatiramer acetate 5-20 mg SC once per day;
memantine 5-20 mg oral once per day and interferon-β1a (REBIF™) 11-44 μg SC three times per week;
rimantadine 50-200 mg oral once per day and interferon-β1a (REBIF™) 11-44 μg SC three times per week;
amantadine 50-200 mg oral once per day and interferon-β1a (REBIF™) 11-44 μg SC three times per week;
memantine 5-20 mg oral once per day and interferon-β1b 50-250 μg SC every other day;
rimantadine 50-200 mg/kg oral once per day and interferon-β1b 50-250 μg SC every other day;
amantadine 50-200 mg oral once per day and interferon-β1b 50-250 μg SC every other day;
memantine 5-20 mg oral once per day and natalizumab [1.5-6 mg/kg] IV infusion at the beginning of the study;
rimantadine 50-200 mg oral once per day and natalizumab [1.5-6 mg/kg] IV infusion at the beginning of the study;
amantadine 50-200 mg oral once per day and natalizumab [1.5-6 mg/kg] IV infusion at the beginning of the study;
memantine 5-20 mg oral once per day and daclizumab [0.25-1 mg/kg] IV infusion at the beginning of the study and at day 14;
rimantadine 50-200 mg oral once per day and daclizumab [0.25-1 mg/kg] IV infusion at the beginning of the study and at day 14;
amantadine 50-200 mg oral once per day and daclizumab [0.25-1 mg/kg] IV infusion at the beginning of the study and at day 14.

### Example 8

### Release Profile of Memantine and Atorvastatin

Release proportions are shown in the tables below for a combination of memantine and atorvastatin. The cumulative fraction is the amount of drug substance released from the formulation matrix to the serum or gut environment (e.g., U.S. Pat. No. 4,839,177) or as measured with a USP II Paddle system using water as the dissolution medium.

| | MEMANTINE | ATORVASTATIN |
|---|---|---|
| | T½ = 60 hrs | T½ = 14 hrs |
| Time | cum. fraction A | cum. fraction B |
| 1 | 0.15 | 0.15 |
| 2 | 0.30 | 0.30 |
| 4 | 0.45 | 0.45 |
| 8 | 0.60 | 0.60 |
| 12 | 0.75 | 0.75 |
| 16 | 0.90 | 0.90 |
| 20 | 0.98 | 0.98 |
| 24 | 0.99 | 0.99 |

### Example 9

### Tablet Containing a Combination of Memantine and Tizanadine Hydrochloride

An extended release dosage form for administration of memantine and tizanadine hydrochloride is prepared as three individual compartments. Three individual compressed tablets are prepared, each having a different release profile, are encapsulated into a gelatin capsule which is then closed and sealed. The components of the three tablets are as follows.

| | | Amount per | |
|---|---|---|---|
| Component | Function | tablet | |
| TABLET 1 (immediate release): | | | |
| Memantine | Active agent | 0 | mg |
| Tizanidine HCl | Active agent | 4.0 | mg |
| Dicalcium phosphate dihydrate | Diluent | 23.6 | mg |
| Microcrystalline cellulose | Diluent | 26.6 | mg |
| Sodium starch glycolate | Disintegrant | 1.2 | mg |
| Magnesium Stearate | Lubricant | 0.6 | mg |
| TABLET 2 (3-5 hour release): | | | |
| Memantine | Active agent | 10 | mg |
| Tizanidine HCl | Active agent | 4.0 | mg |
| Dicalcium phosphate dihydrate | Diluent | 23.6 | mg |
| Microcrystalline cellulose | Diluent | 26.6 | mg |
| Sodium starch glycolate | Disintegrant | 1.2 | mg |
| Magnesium Stearate | Lubricant | 0.6 | mg |
| Eudragit RS30D | Delayed release | 4.76 | mg |
| Talc | Coating component | 3.3 | mg |
| Triethyl citrate | Coating component | 0.95 | mg |
| TABLET 3 (Release delayed 7-10 | | | |
| hours): | | | |
| Memantine | Active agent | 12.5 | mg |
| Tizanidine HCl | Active agent | 4.0 | mg |
| Dicalcium phosphate dihydrate | Diluent | 23.1 | mg |
| Microcrystalline cellulose | Diluent | 26.6 | mg |
| Sodium starch glycolate | Disintegrant | 1.2 | mg |
| Magnesium Stearate | Lubricant | 0.6 | mg |
| Eudragit RS30D | Delayed release | 6.5 | mg |
| Talc | Coating component | 4.4 | mg |
| Triethyl citrate | Coating component | 1.27 | mg |

The tablets are prepared by wet granulation of the individual drug particles and other core components as may be done using a fluid-bed granulator, or are prepared by direct compression of the admixture of components. Tablet 1 is an immediate release dosage form, releasing the active agents within 1-2 hours following administration. It contains no memantine to avoid the dC/dT effects of the current dosage forms. Tablets 2 and 3 are coated with the delayed release coating material as may be carried out using conventional coating techniques such as spray-coating or the like. The specific components listed in the above tables may be replaced with other functionally equivalent components, e.g., diluents, binders, lubricants, fillers, coatings, and the like.

Oral administration of the capsule to a patient will result in a release profile having three pulses, with initial release of tizanidine HCl from the first tablet being substantially immediate, release of the memantine and tizanidine HCl from the second tablet occurring 3-5 hours following administration, and release of the memantine and tizanidine HCl from the third tablet occurring 7-9 hours following administration.

### Example 10

### Beads Containing a Combination of Memantine and Doxycycline

The method of Example 9 is repeated substituting doxycycline for tizanidine HCl and using drug-containing beads in place of tablets. A first fraction of beads is prepared by coating an inert support material such as lactose with the drug which provides the first (immediate release) pulse. A second fraction of beads is prepared by coating immediate release beads with an amount of enteric coating material sufficient to provide a drug release-free period of 3-7 hours. A third fraction of beads is prepared by coating immediate release beads having half the ibuprofen dose of the first fraction of beads with a greater amount of enteric coating material, sufficient to provide a drug release-free period of 7-12 hours. The quantities of doxycycline in each of the bead types is adjusted from the previous example to provide in a unit dose of 60 mg doxycycline evenly divided among the three types of beads. The three groups of beads may be encapsulated as in Example 9, or compressed, in the presence of a cushioning agent, into a single pulsatile release tablet. Alternatively, three groups of drug particles may be provided and coated as above, in lieu of the drug-coated lactose beads.

### Example 11

### Dissolution and Plasma Profiles

Experimental dissolution profiles were obtained from a USP II Paddle system using water as the medium. Simulations for tizanidine HCl and doxycycline were generated using the Gastro Plus Software Package v.4.0.2. The corresponding in vivo release profiles were obtained using the Gastro-Plus software package v.4.0.2.

Summary Table of PK Values

| | | SINGLE | | | |
|---|---|---|---|---|---|
| | | DOSE | AUC | | |
| | Tmax | Cmax | (ng- | dC/dT | |
| Formulation | (hr) | (ug/mL) | h/mL) | (*1e5) | % BA |
| Namenda (5 mg) | 6.2 | 0.005972 | 628.64 | 9.63 | 99.2 |
| Namenda (20 mg) | 6.2 | 0.02389 | 2514.5 | 38.53 | 99.2 |
| Namenda (10 mg) | 6.2 | 0.01194 | 1257.3 | 19.26 | 99.2 |
| 5001-6601 (22.5 mg) | 17.3 | 0.02366 | 2691.4 | 13.68 | 94.5 |
| 5001-6701 (22.5 mg) | 20.4 | 0.02261 | 2546 | 11.08 | 89.5 |
| 5001-6801 (22.5 mg) | 25.1 | 0.02324 | 2324 | 12.17 | 81.9 |
| Tizanidine IR (8 mg) | 1.2 | 0.00406 | 11.87 | 33.83 | 5 |
| Tizanidine | 2.4 | 0.00298 | 18.3 | 12.42 | 4.7 |
| SR Fast (13 mg) | | | | | |
| Tizanidine | 3.66 | 0.00141 | 17.1 | 3.85 | 4.4 |
| SR Medium (13 mg) | | | | | |
| Doxycycline | 1.9 | 2.67 | 5458 | 14052.63 | 98.6 |
| IR (200 mg) | | | | | |
| Doxycycline | 3.8 | 1.99 | 5452 | 5236.84 | 82.3 |
| SR Fast (240 mg) | | | | | |
| Doxycycline | 4 | 1.4 | 4813 | 3500.00 | 72.8 |
| SR Medium (240 mg) | | | | | |
| Doxycycline | 23.3 | 0.8 | 3647 | 343.35 | 54 |
| SR Slow (240 mg) | | | | | |

dC/dT = Cmax/Tmax
BOLD = The dC/dT was adjusted for dissolution lag. (lag value is based on time at Cp = 0)

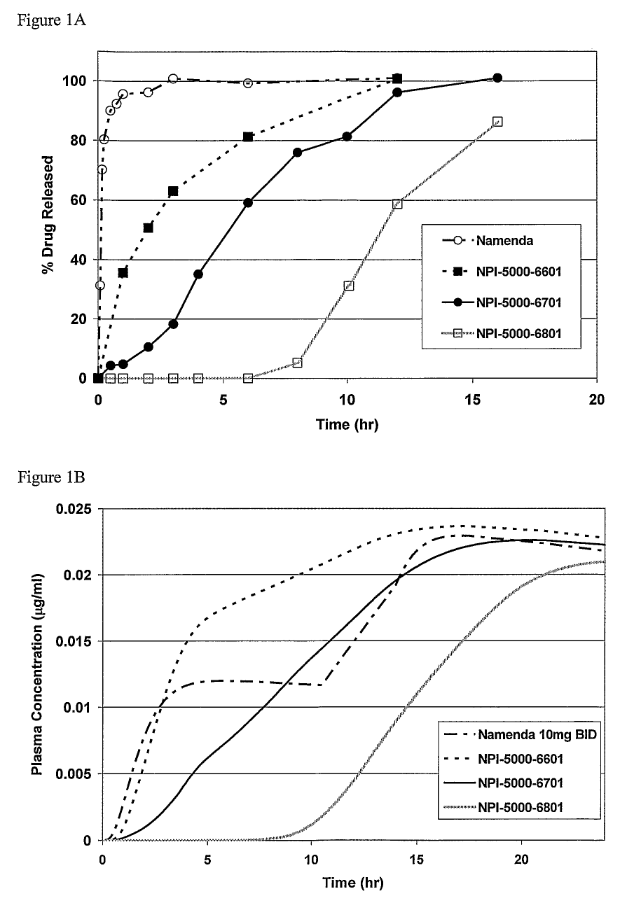

Memantine Component of the Matrix Tablet Formulation 6601 Shown in FIGS. 1A and 1B.

| | |
|---|---|
| Memantine HCL (22.5 mg) | 13.51% |
| Avicel PH102 | 60.04% |
| Eudragit RS-30D (30% w/w | 15.37% |
| aqueous dispersion) | |
| HPMC K100M | 10.08% |
| Magnesium Stearate | 1.00% |
| Total Component Weight | 166.5 mg |

Memantine Component of the Coated Tablet Formulation 6701 Shown in FIGS. 1A and 1B.

| | |
|---|---|
| Memantine HCL (22.5 mg) | 13.21% |
| Avicel PH102 | 58.72% |
| Eudragit RS-30D (30% w/w | 15.03% |
| aqueous dispersion) | |
| HPMC K100M | 9.86% |
| Magnesium Stearate | 0.98% |
| Opadry ® Clear, (Formulation | 2.20% |
| YS-1-7006, Colorcon) | |
| Total Component Weight | 170.3 mg |

Memantine Component of the Coated Tablet Formulation 6801 Shown in FIGS. 1A and 1B.

| | |
|---|---|
| Memantine HCL (22.5 mg) | 12.77% |
| Avicel PH102 | 56.55% |
| Eudragit RS-30D (30% w/w | 14.48% |
| aqueous dispersion) | |
| HPMC K100M | 9.50% |
| Magnesium Stearate | 0.94% |
| Opadry ® Clear, (Formulation | 3.00% |
| YS-1-7006, Colorcon) | |
| Surelease ® Clear, (Formulation | 2.80% |
| E-7-19010, Colorcon) | |
| Total Component Weight | 176.2 mg |

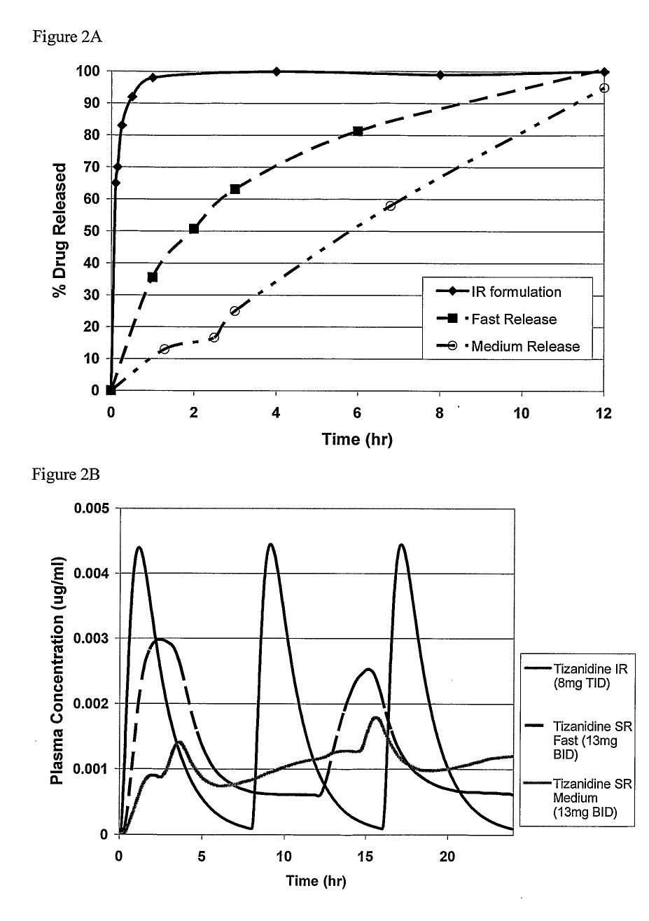

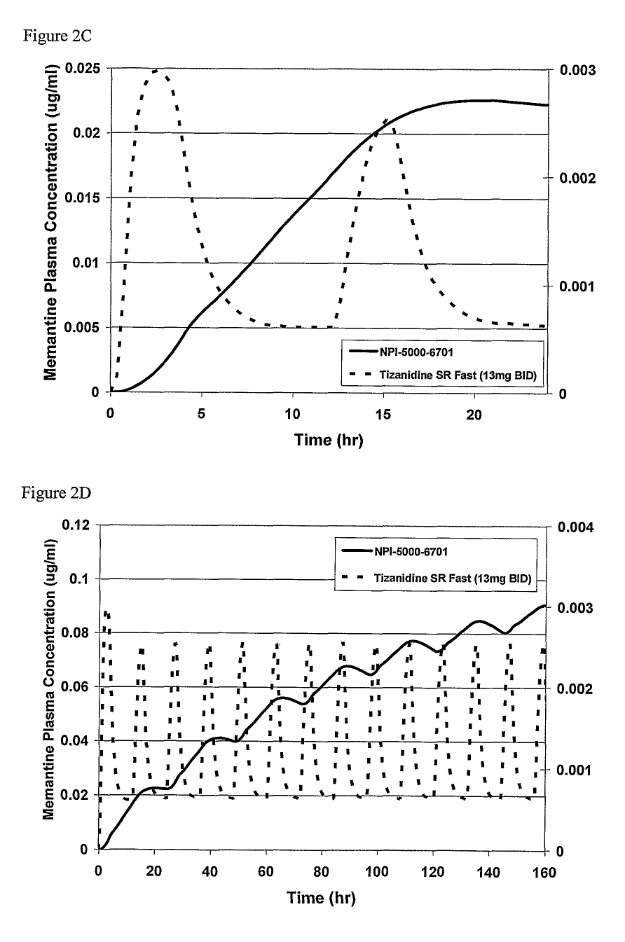

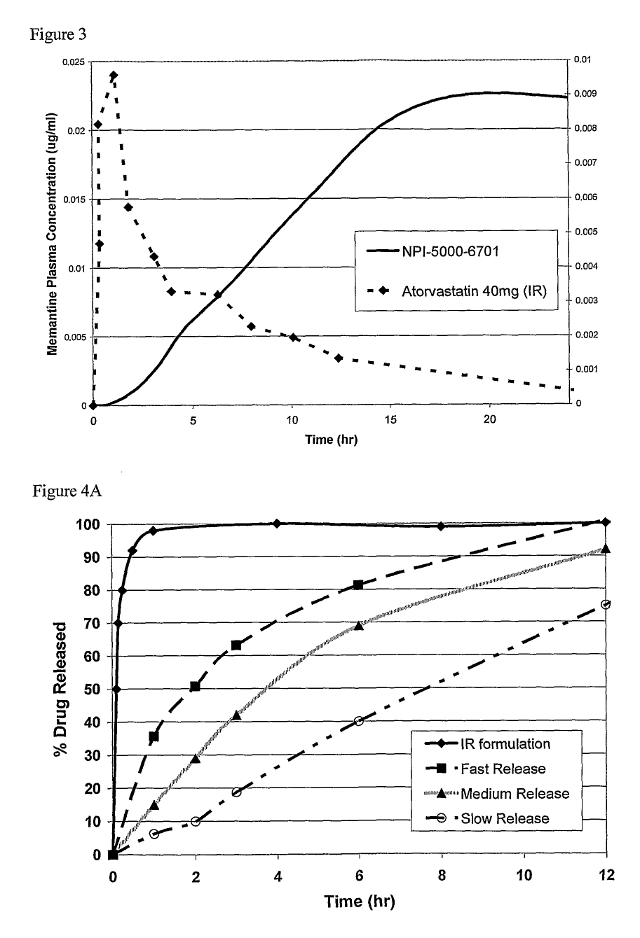

Tizanidine HCl Component of the Matrix Tablet Formulation Short Shown in FIGS. 2A-2D.

| | |
|---|---|
| Tizanidine HCl (120 mg) | 13.56% |
| Avicel PH102 | 60.04% |
| Eudragit RS-30D (30% w/w | 15.37% |
| aqueous dispersion) | |
| HPMC K100M | 10.08% |
| Magnesium Stearate | 1.00% |
| Total Component Weight | 885 mg |

Tizanidine HCl Component of the Coated Tablet Formulation SR Shown in FIGS. 2A-2D.

| | |
|---|---|
| Tizanidine HCl (120 mg) | 13.21% |
| Avicel PH102 | 58.72% |
| Eudragit RS-30D (30% w/w | 15.03% |
| aqueous dispersion) | |
| HPMC K100M | 9.86% |
| Magnesium Stearate | 0.98% |
| Opadry ® Clear, (Formulation | 2.20% |
| YS-1-7006, Colorcon) | |
| Total Component Weight | 908.4 mg |

Tizanidine HCl Component of the Coated Tablet Formulation Long Shown in FIGS. 2A-2D.

| | |
|---|---|
| Tizanidine HCl (120 mg) | 12.77% |
| Avicel PH102 | 56.55% |
| Eudragit RS-30D (30% w/w | 14.48% |
| aqueous dispersion) | |
| HPMC K100M | 9.50% |
| Magnesium Stearate | 0.94% |
| Opadry ® Clear, (Formulation | 3.00% |
| YS-1-7006, Colorcon) | |
| Surelease ® Clear, (Formulation | 2.80% |
| E-7-19010, Colorcon) | |
| Total Component Weight | 940 mg |

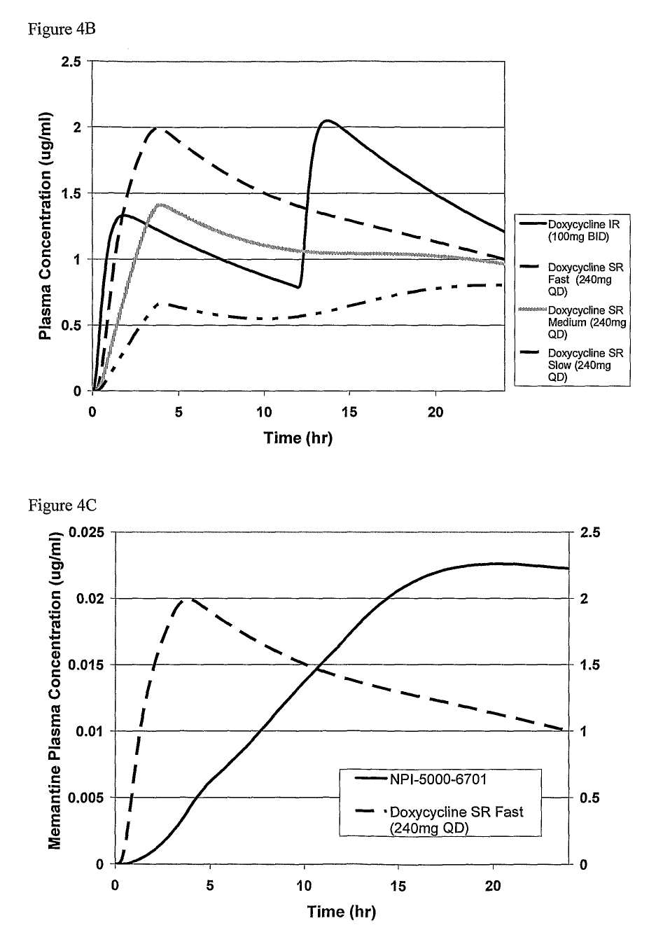

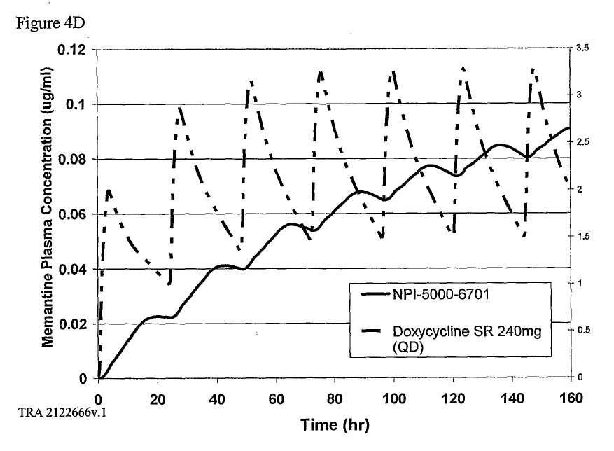

Doxycycline Component of the Matrix Tablet Formulation SR Shown in FIGS. 4A-4D.

| | |
|---|---|
| Doxycycline (25 mg) | 20.83.% |
| Avicel PH102 | 37.28% |
| Eudragit RS-30D (30% w/w | 15.37% |
| aqueous dispersion) | |
| HPMC K100M | 10.08% |
| Magnesium Stearate | 1.00% |
| Total Component Weight | 120 mg |

Doxycycline Component of the Coated Tablet Formulation Short Shown in FIGS. 4A-4D.

| | |
|---|---|
| Doxycycline (25 mg) | 20.83% |
| Lactose NF | 29.17% |
| Microcrystalline Cellulose NF | 39.80% |
| HPMC K100M | 8.20% |
| Magnesium Stearate | 1.00% |
| Opadry ® Clear, (Formulation YS- | 1.00% |
| 1-7006, Colorcon) | |
| Total Component Weight | 120 mg |

Doxycycline Component of the Coated Tablet Formulation Linear Shown in FIGS. 4A-4D.

| | |
|---|---|
| Doxycycline (25 mg) | 19.17% |
| Avicel PH102 | 52.76% |
| Eudragit RS-30D (30% w/w | 15.03% |
| aqueous dispersion) | |
| HPMC K100M | 9.86% |
| Magnesium Stearate | 0.98% |
| Opadry ® Clear, (Formulation | 2.20% |
| YS-1-7006, Colorcon) | |
| Total Component Weight | 130.4 mg |

Doxycycline Component of the Coated Tablet Formulation Long Shown in FIGS. 4A-4D.

| | |
|---|---|
| Doxycycline (25 mg) | 18.17% |
| Avicel PH102 | 51.11% |
| Eudragit RS-30D (30% w/w | 14.48% |
| aqueous dispersion) | |
| HPMC K100M | 9.50% |
| Magnesium Stearate | 0.94% |
| Opadry ® Clear, (Formulation | 3.00% |
| YS-1-7006, Colorcon) | |
| Surelease ® Clear, (Formulation | 2.80% |
| E-7-19010, Colorcon) | |
| Total Component Weight | 137.6 mg |

### Example 12

### A Patch Providing Extended Release of Memantine and Tizanidine HCl

As described above, extended release formulations of an NMDA antagonist are formulated for topical administration. Memantine transdermal patch formulations are prepared as described, for example, in U.S. Pat. Nos. 6,770,295 and 6,746,689.

For the preparation of a drug-in-adhesive acrylate, 5 g of memantine and 1 g of tizanidine HCl are dissolved in 10 g of ethanol and this mixture is added to 20 g of Durotak 387-2287 (National Starch & Chemical, U.S.A.). The drug gel is coated onto a backing membrane (Scotchpak 1012; 3M Corp., U.S.A.) using a coating equipment (e.g., RK Print Coat Instr. Ltd, Type KCC 202 control coater). The wet layer thickness is 400 μm. The laminate is dried for 20 minutes at room temperature and then for 30 minutes at 40° C. A polyester release liner is laminated onto the dried drug gel. The sheet is cut into patches and stored at 2-8° C. until use (packed in pouches). The concentration of memantine in the patches ranges between 5.6 and 8 mg/cm², while tizanidine HCl ranges between 1.1 and 1.6 mg/cm². The nearly continuous infusion of the components provides a much more consistent Cratio over time maximizing the additive or synergistic effects of the combinations of the present invention to achieve the optimal therapeutic effects.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby fully incorporated by reference. The appropriate components, processes, and methods of those patents, applications and other documents may be selected for the present invention and embodiments thereof.

What is claimed is:

1. A pharmaceutical composition comprising:
(a) an uncompetitive NMDA receptor antagonist;
(b) a second agent, wherein said second agent is a multiple sclerosis agent; and
(c) a pharmaceutically acceptable carrier.

2. The pharmaceutical composition of claim 1, wherein at least one of said NMDA receptor antagonist or said second agent is provided in an extended release dosage form.

3. The pharmaceutical composition of claim 1, wherein said NMDA receptor antagonist has a dC/dT less than about 80% of the rate for the IR formulation.

4. The pharmaceutical composition of claim 1, wherein said NMDA receptor antagonist has a Cₘₐₓ/Cₘₑₐₙ of approximately 1.6 or less approximately 2 hours to at least 12 hours after said composition is introduced into a subject.

5. The pharmaceutical composition of claim 1, wherein the relative Cratio.var of said NMDA receptor antagonist and said second agent is less than 100% from 2 hour to 12 hours after said composition is introduced into a subject.

6. The pharmaceutical composition of claim 1, wherein the relative Cratio.var of said NMDA receptor antagonist and said second agent is less than 70% of the corresponding IR formulation from 2 hour to 12 hours after said composition is introduced into a subject.

7. The pharmaceutical composition of claim 1, wherein the said uncompetitive NMDA receptor antagonist is selected from the group consisting of memantine, rimantadine, and amantadine, and pharmaceutically acceptable salts thereof.

8. The pharmaceutical composition of claim 1, wherein said multiple sclerosis agent is selected from the group consisting of β-interferons, glatiramer acetate, natalizumab, mitoxanthrone, mycophenolic acid, tizanidine, atorvastatin, and daclizumab.

9. The pharmaceutical composition of claim 8, wherein said multiple sclerosis agent is glatiramer acetate (COPAXONE™) administered subcutaneously at a dosage of from about 5 mg to about 20 mg/day.

10. The pharmaceutical composition of claim 8, wherein said multiple sclerosis agent is natalizumab.

11. The pharmaceutical composition of claim 8, wherein said multiple sclerosis agent is daclizumab.

12. The pharmaceutical composition of claim 8, wherein said multiple sclerosis agent is mitoxanthrone (NOVANTRONE™) administered at a dose of from about 3 mg/m² to about 12 mg/m² for about 5 to 15 minutes intravenously.

13. The pharmaceutical composition of claim 7, wherein the said uncompetitive NMDA receptor antagonist is memantine.

14. The pharmaceutical composition of claim 13, wherein said memantine is administered at a dose of about 5 to 80 mg/day.

15. The pharmaceutical composition of claim 14, wherein the said uncompetitive NMDA receptor antagonist is administered at a dose of about 10 to 40 mg/day.

16. A method of treating a demyelinating condition or a symptom thereof comprising administering to a subject in need thereof a therapeutically effective amount of:
(a) an uncompetitive NMDA receptor antagonist; and
(b) a second agent, wherein said second agent is a multiple sclerosis agent, such that said demyelinating condition or a symptom thereof is treated or at least partially alleviated.

17. The method of claim 16, wherein the amount of said uncompetitive NMDA receptor channel antagonist and/or said a multiple sclerosis agent is effective to reduce symptoms and to enable an observation of a reduction in symptoms.

18. The method of claim 16, wherein said demyelinating condition is selected from the group consisting of multiple sclerosis (MS); progressive multifocal leukoencephalopathy (PML); disseminated necrotizing leukoencephalopathy (DNL); acute disseminated encephalomyelitis; Schilder disease, central pontine myelinolysis (CPM); radiation necrosis; Binswanger disease(SAE); Guillain-Barre Syndrome; leukodystrophy; acute disseminated encephalomyelitis (ADEM); acute transverse myelitis; acute viral encephalitis; adrenoleukodystrophy (ALD); adrenomyeloneuropathy; AIDS-vacuolar myelopathy; experimental autoimmune encephalomyelitis (EAE); experimental autoimmune neuritis (EAN); HTLV-associated myelopathy; Leber's hereditary optic atrophy; subacute sclerosing panencephalitis; and tropical spastic paraparesis.

19. The method of claim 16, wherein said demyelinating condition is multiple sclerosis.

20. The method of claim 16, wherein at least one of said NMDA receptor antagonist or said second agent is provided in an extended release dosage form.

21. The method of claim 16, wherein said NMDA receptor antagonist is provided in an extended release dosage form.

22. The method of claim 21, wherein said NMDA receptor antagonist is administered at a substantially identical daily dose.

23. The method of claim 22, wherein said NMDA receptor antagonist reaches a therapeutically effective steady state plasma concentration in a subject within fifteen days of said administering.

24. The method of claim 21, wherein said NMDA receptor antagonist has a dC/dT less than about 80% of the rate for the IR formulation.

25. The method of claim 21, wherein the relative Cratio.var of said NMDA receptor antagonist and said second agent is less than 100% from 2 hour to 12 hours after said composition is introduced into a subject.

26. The method of claim 16, wherein said uncompetitive NMDA receptor channel antagonist is selected from the group consisting of memantine, rimantadine, and amantadine.

27. The method of claim 16, wherein said multiple sclerosis agent is selected from the group consisting of β-interferons, glatiramer acetate, natalizumab, mitoxanthrone, mycophenolic acid, tizanidine, atorvastatin, and daclizumab.

28. The method of claim 16, wherein said multiple sclerosis agent is glatiramer acetate (COPAXONE™) administered subcutaneously at a dosage of from about 5 mg to about 20 mg per day.

29. The method of claim 16, wherein said multiple sclerosis agent is natalizumab.

30. The method of claim 16, wherein said multiple sclerosis agent is daclizumab.

31. The method of claim 16, wherein said NMDA receptor channel antagonist is memantine.

32. The method of claim 31, wherein the dose of memantine is at least about 5 to 80 mg per day.

33. The method of claim 32, wherein the dose of memantine is at least about 10 to 40 mg per day.

34. A kit comprising:
(a) an NMDA receptor antagonist;
(b) a second agent, wherein said second agent is a multiple sclerosis agent; and
(c) instructions for treating or partially alleviating a demyelinating condition.

35. A method of treating a symptom associated with multiple sclerosis comprising administering to a subject having multiple sclerosis a combination therapy including a memantine and a β-interferon, such that said symptom associated with multiple sclerosis is treated or at least partially alleviated.

36. The method of claim 35, wherein the symptom is selected from the group consisting of fatigue, pain and tingling in the arms and legs; localized and generalized numbness, muscle spasm and weakness; bowel and bladder dysfunction; and difficulty with balance when walking or standing.
